(19) United States
(12) Patent Application Publication
(10) Pub. No.: US 2022/0298241 A1
(43) Pub. Date: Sep. 22, 2022

(54) USE OF ANTI-FCRN ANTIBODIES IN THE TREATMENT OF PEMPHIGHUS AND PEMPHIGOID DISEASES

(71) Applicant: Alexion Pharmaceuticals, Inc., Boston, MA (US)

(72) Inventors: Laurence J. Blumberg, New York, NY (US); Richard S. Blumberg, Weston, MA (US); John Humphries, Charlottesville, VA (US)

(73) Assignee: Alexion Pharmaceuticals, Inc., Boston, MA (US)

(21) Appl. No.: 17/611,656

(22) PCT Filed: May 18, 2020
(86) PCT No.: PCT/US20/33366

§ 371 Date: Nov. 16, 2021

Related U.S. Application Data
(60) Provisional application No. 62/849,188, filed on May 17, 2019.
(60) Provisional application No. 62/890,775, filed on Aug. 23, 2019.
(60) Provisional application No. 62/958,973, filed on Jan. 9, 2020.

(51) Int. Cl. C07K 16/28; A61P 37/06; A61P 17/00; G01N 33/564

(52) U.S. Cl. CPC C07K 16/283; A61P 37/06; A61P 17/00; G01N 33/564; A61K 2039/505

(57) ABSTRACT

The disclosure relates to methods for treating pemphigus and/or a pemphigoid disease in a subject in need thereof, wherein the methods include administering to a subject in need thereof a therapeutically effective amount of an FcRn inhibitor. In certain embodiments, the FcRn inhibitor is an anti-FcRn antibody or antigen-binding fragment thereof.

### FIELD OF THE INVENTION

The invention relates to methods for treating phemphigus and/or a pemphigoid disease by administering a neonatal Fc receptor (FcRn) inhibitor, including, but not limited to, an antibody or antigen-binding fragment thereof that binds to FcRn.

### BACKGROUND

Pemphigus and pemphigoid diseases are autoimmune blistering diseases of the skin and/or mucous membranes. Pemphigus affects the outer of the skin (epidermis) and causes lesions and blisters that are easily ruptured. Pemphigoid affects a lower layer of the skin, between the epidermis and the dermis, creating tense blisters that do not break easily. The prognosis of pemphigus has markedly improved over the last decades with steroid therapy. Nevertheless, mortality remains an issue (1.6% to 12% of cases) (Hsu et al., Br J Dermatol. 2016; 174(6):1290-8; Kasperkiewicz et al., Nat Rev Dis Primers. 2017; 3:17026; Langan et al., BMJ. 2008; 337:a180). In these cases, death typically occurs as a consequence of treatment-related systemic infections and in a smaller proportion, as a consequence of superinfected lesions.

While steroids have greatly improved outcomes for patients with pemphigus or pemphigoid diseases, steroids are associated with serious and long-lasting side effects; therefore, the use of steroids should be limited as much as possible. Although other currently available treatments for certain autoimmune disorders, including immunosuppressants, intravenous immunoglobulin (WIG), plasmapheresis, and anti-CD20 monoclonal antibodies (mAbs), such as rituximab, can be effective, they can be associated with significant adverse effects and delayed or non-durable responses.

As such, new methods for the treatment of pemphigus and pemphigoid diseases are needed.

### SUMMARY OF THE INVENTION

The present invention relates to methods for treating pemphigus and/or pemphigoid diseases.

In one aspect, provided is a method of treating pemphigus and/or a pemphigoid disease in a subject in need thereof, the method comprising administering to the subject an FcRn inhibitor, wherein the FcRn inhibitor is administered at a dose of at least 10 mg/kg of the subject's body weight. In one embodiment, the FcRn inhibitor is administered at a dose of at least 10 mg/kg of the subject's body weight once a week for at least five weeks. In one embodiment, the FcRn inhibitor is administered at a dose of 10 mg/kg of the subject's body weight. In one embodiment, the FcRn inhibitor is administered once a week for five weeks. In one embodiment, the FcRn inhibitor is administered at a dose of 10 mg/kg of the subject's body weight once a week for five weeks.

Provided herein are methods of treating pemphigus and/or a pemphigoid disease in a subject in need thereof, wherein the pemphigus is pemphigus vulgaris, pemphigus foliaceus, paraneoplastic pemphigus, drug-induced pemphigus, endemic pemphigus (fogo selvagem), pemphigus erythematosus (Senear-Usher syndrome), or pemphigus vegetans. In one embodiment, the pemphigoid disease is bullous pemphigoid, mucous membrane pemphigoid, pemphigoid gestationis, epidermolysis bullosa acquisita, anti-laminin g1/anti-p200 pemphigoid, or lichen planus pemphigoid. In one embodiment, the pemphigus is pemphigus foliaceus. In one embodiment, the pemphigus is pemphigus vulgaris.

Provided herein are methods of treating pemphigus and/or a pemphigoid disease in a subject in need thereof, wherein the subject: (1) has been diagnosed with pemphigus vulgaris or foliaceus based on (i) a clinical history consistent with pemphigus vulgaris or foliaceus, (b) presence of anti-Dsg 1 or anti-Dsg3 antibodies above the upper limit of normal, and/or (c) a history of at least one positive tissue-based test for pemphigus vulgaris or foliaceus; (2) experiences active pemphigus vulgaris or foliaceus and has (i) lesions lasting longer than two weeks, and/or (ii) at least three active lesions in skin or mucosa or at least two active lesions, wherein at least one of the at least two active lesions is a skin lesion with a diameter of at least 1 cm; and/or (3) exhibits a Pemphigus Disease Area Index (PDAI) total activity score of at least four.

Provided herein are methods of treating pemphigus and/or a pemphigoid disease in a subject in need thereof, the method further comprising: (a) measuring a level of IgG for the subject, wherein administering the FcRn inhibitor leads to a decrease in IgG level; (b) measuring a level of circulating immune complexes (CIC) for the subject, wherein administering the FcRn inhibitor leads to a decrease in CIC level; (c) measuring the PDAI total activity score for the subject, wherein administering the FcRn inhibitor leads to a decrease in PDAI total activity score; (d) measuring an anti-Dsg1 antibody titer for the subject, wherein administering the FcRn inhibitor leads to a decrease in anti-Dsg1 antibody titer; (e) measuring an anti-Dsg3 antibody titer for the subject, wherein administering the FcRn inhibitor leads to a decrease in anti-Dsg3 antibody titer; (f) measuring an anti-epithelial cell antibody (AECA) titer for the subject, wherein administering the FcRn inhibitor leads to a decrease in AECA titer; or (g) measuring a complement component 3 (C3) level for the subject, wherein administering the FcRn inhibitor leads to a decrease in the C3 level.

Provided herein are methods of treating pemphigus and/or a pemphigoid disease in a subject in need thereof, the wherein the subject exhibits one or more of the following conditions and wherein the administration of the FcRn inhibitor reduces the occurrence of one or more of the following conditions: (a) fluid-filled skin blisters; (b) ruptured blisters; (c) scaly, inflamed, painful patches on the skin; (d) burning, pain, and itching at the site of the blisters; and/or (e) chronic skin infections due to ruptured and irritated blisters.

Provided herein is a method of treating pemphigus and/or a pemphigoid disease in a subject in need thereof, the method comprising administering to the subject an FcRn inhibitor, wherein the FcRn inhibitor is an anti-FcRn antibody or antigen-binding fragment thereof, wherein the anti-FcRn antibody or antigen-binding fragment thereof comprises a heavy chain variable region comprising a CDR1, CDR2, and CDR3 (HCDR1, HCDR2 and HCDR3) and a light chain variable region comprising a CDR1, CDR2, and CDR3 (LCDR1, LCDR2 and LCDR3); and wherein: (a) HCDR1 comprises the amino acid sequence of SEQ ID NO: 3; HCDR2 comprises the amino acid sequence of SEQ ID NO: 4; HCDR3 comprises the amino acid sequence of SEQ ID NO: 5; LCDR1 comprises the amino acid sequence of SEQ ID NO: 6; LCDR2 comprises the amino acid sequence of SEQ ID NO: 7; and LCDR3 comprises the amino acid sequence of SEQ ID NO: 8; (b) HCDR1 comprises the amino acid sequence of SEQ ID NO: 11 or SEQ ID NO: 12; HCDR2 comprises the amino acid sequence of SEQ ID NO: 13 or SEQ ID NO: 14; HCDR3 comprises the amino acid sequence of SEQ ID NO: 15, SEQ ID NO: 16, SEQ ID NO: 17, SEQ ID NO: 18, or SEQ ID NO: 19; LCDR1 comprises the amino acid sequence of SEQ ID NO: 20; LCDR2 comprises the amino acid sequence of SEQ ID NO: 21; and LCDR3 comprises the amino acid sequence of SEQ ID NO: 22; (c) HCDR1 comprises the amino acid sequence of SEQ ID NO: 11; HCDR2 comprises the amino acid sequence of SEQ ID NO: 13; HCDR3 comprises the amino acid sequence of SEQ ID NO: 19; LCDR1 comprises the amino acid sequence of SEQ ID NO: 20; LCDR2 comprises the amino acid sequence of SEQ ID NO: 21; and LCDR3 comprises the amino acid sequence of SEQ ID NO: 23 or SEQ ID NO: 24; (d) HCDR1 comprises the amino acid sequence of SEQ ID NO: 25; HCDR2 comprises the amino acid sequence of SEQ ID NO: 26; HCDR3 comprises the amino acid sequence of SEQ ID NO: 27; LCDR1 comprises the amino acid sequence of SEQ ID NO: 28; LCDR2 comprises the amino acid sequence of SEQ ID NO: 29; and LCDR3 comprises the amino acid sequence of SEQ ID NO: 30; (e) HCDR1 comprises the amino acid sequence of SEQ ID NO: 31; HCDR2 comprises the amino acid sequence of SEQ ID NO: 32; HCDR3 comprises the amino acid sequence of SEQ ID NO: 33; LCDR1 comprises the amino acid sequence of SEQ ID NO: 34; LCDR2 comprises the amino acid sequence of SEQ ID NO: 35; and LCDR3 comprises the amino acid sequence of SEQ ID NO: 36; (f) HCDR1 comprises the amino acid sequence of SEQ ID NO: 37; HCDR2 comprises the amino acid sequence of SEQ ID NO: 38; HCDR3 comprises the amino acid sequence of SEQ ID NO: 39; LCDR1 comprises the amino acid sequence of SEQ ID NO: 40; LCDR2 comprises the amino acid sequence of SEQ ID NO: 41; and LCDR3 comprises the amino acid sequence of SEQ ID NO: 42; or (g) HCDR1 comprises the amino acid sequence of SEQ ID NO: 43; HCDR2 comprises the amino acid sequence of SEQ ID NO: 44; HCDR3 comprises the amino acid sequence of SEQ ID NO: 19; LCDR1 comprises the amino acid sequence of SEQ ID NO: 20; LCDR2 comprises the amino acid sequence of SEQ ID NO: 45; and LCDR3 comprises the amino acid sequence of SEQ II) NO; 23.

In one embodiment, provided is a method of treating pemphigus and/or a pemphigoid disease in a subject in need thereof, the method comprising administering to the subject an FcRn inhibitor, wherein the FcRn inhibitor is an anti-FcRn antibody or antigen-binding fragment thereof, wherein the anti-FcRn antibody or antigen-binding fragment thereof comprises a heavy chain variable region comprising a CDR1, CDR2, and CDR3 (HCDR1, HCDR2 and HCDR3) and a light chain variable region comprising a CDR1, CDR2, and CDR3 (LCDR1, LCDR2 and LCDR3); and wherein the HCDR1 comprises the amino acid sequence of SEQ ID NO: 3; HCDR2 comprises the amino acid sequence of SEQ ID NO: 4; HCDR3 comprises the amino acid sequence of SEQ ID NO: 5; LCDR1 comprises the amino acid sequence of SEQ ID NO: 6; LCDR2 comprises the amino acid sequence of SEQ ID NO: 7; and LCDR3 comprises the amino acid sequence of SEQ ID NO: 8.

In one embodiment, the FcRn inhibitor is an anti-FcRn antibody or antigen-binding fragment thereof comprising a heavy chain variable region and a light chain variable region, wherein: (1) the heavy chain variable region comprises the sequence of SEQ ID NO: 1, or a sequence that is at least 80% identical to the sequence of SEQ ID NO: 1; and (2) the light chain variable region comprises the sequence of SEQ ID NO: 2, or a sequence that is at least 80% identical to the sequence of SEQ ID NO: 2. In one embodiment, the FcRn inhibitor is an anti-FcRn antibody or antigen-binding fragment thereof comprising a heavy chain variable region and a light chain variable region, wherein the heavy chain variable region comprises the sequence of SEQ ID NO: 1 and the light chain variable region comprises the sequence of SEQ ID NO: 2.

In one embodiment, the FcRn inhibitor is an anti-FcRn antibody or antigen-binding fragment thereof comprising a heavy and a light chain, wherein: (1) the heavy chain comprises the amino acid sequence of SEQ ID NO: 9, or a sequence that is at least 80% identical to a sequence of SEQ ID NO:9; and (2) the light chain comprises the amino acid sequence of SEQ ID NO: 10, or a sequence that is at least 80% identical to a sequence of SEQ ID NO: 10. In one embodiment, the FcRn inhibitor is an anti-FcRn antibody or antigen-binding fragment thereof comprising a heavy and a light chain, wherein the heavy chain comprises the amino acid sequence of SEQ ID NO: 9 and the light chain comprises the amino acid sequence of SEQ ID NO: 10.

Provided herein is a method of treating pemphigus and/or a pemphigoid disease in a subject in need thereof, the method comprising administering to the subject an FcRn inhibitor, wherein the FcRn inhibitor is an Fc region, or FcRn-binding fragment thereof, and wherein the Fc region comprises the amino acid sequence of SEQ ID NO:46, SEQ ID NO:47, or SEQ ID NO:48.

### BRIEF DESCRIPTION OF THE DRAWINGS

FIG. 1A and FIG. 1B show the mean serum concentration-time profiles (linear scale and semi-logarithmic scale) following 1-hour infusion of 10 mg/kg of the study drug on Day 0 (FIG. 1A) and Day 28 (FIG. 1B). For calculation of mean concentrations and generation of mean concentration-time profiles, all below the limit of quantification (125 ng/mL) values were set to 0 except when an individual BLQ (below the limit of quantification) fell between 2 quantifiable values, in which case it was omitted. The pharmacokinetic population consisted of all subjects who received at least 1 dose of study drug and had sufficient post-dose blood samples to obtain pharmacokinetic parameters. Actual sampling times that were outside the scheduled sampling times window were excluded from the figures.

FIG. 2 shows the percentage change (±SD) from baseline for the serum total IgG levels. Baseline was defined as Day 0 visit (pre-dose) measurement. If missing, the last measurement prior to the first study drug administration was used.

FIG. 3 shows the percentage change (±SD) from baseline for circulating immune complex (CIC) levels as determined by CIC-serum C1Q binding assay. Baseline was defined as Day 0 visit (pre-dose) measurement. If missing, the last measurement prior to the first study drug administration was used.

FIG. 4 shows the percentage change (±SD) from baseline for anti-desmoglein 1 (anti-Dsg1) antibody levels. Baseline was defined as Day 0 visit (pre-dose) measurement. If missing, the last measurement prior to the first study drug administration was used.

FIG. 5 shows the percentage change (±SD) from baseline for anti-desmoglein 3 (anti-Dsg3) antibody levels. Baseline was defined as Day 0 visit (pre-dose) measurement. If missing, the last measurement prior to the first study drug administration was used.

FIG. 6 the percentage change (±SD) from baseline in Pemphigus Disease Area Index (PDAI) total activity score (safety population). Baseline was defined as Day 0 visit (pre-dose) measurement.

### DETAILED DESCRIPTION OF THE INVENTION

It is to be understood that the present invention is not limited to the particular methods and conditions described, as such methods and conditions may vary. It is also to be understood that the terminology used herein is for the purpose of describing particular embodiments only, and is not intended to be limiting.

Unless defined otherwise, all technical and scientific terms used herein have the same meaning as commonly understood by one of ordinary skill in the art to which the disclosed invention belongs.

In one aspect, provided are methods of treating pemphigus and/or a pemphigoid disease, the methods comprising administering an FcRn inhibitor to a subject in need thereof. FcRn inhibitors target key mechanisms contributing to pathology in a variety of immunoglobulin G (IgG)-mediated autoimmune disorders, including IgG-mediated pemphigus and IgG-mediated pemphigoid diseases. FcRn is an intracellular trafficking integral membrane Fc receptor for IgG. While FcRn was originally identified as a receptor functioning in neonatal life, FcRn is today known to continue to function throughout adult life. FcRn resides primarily in the early acidic endosomes where it regulates serum IgG concentrations by binding to and protecting endocytosed monomeric IgG from degradation in the lysosomal compartment, and transporting the IgG to the cell surface for release at neutral extracellular pH. Through this mechanism, FcRn is responsible for the long serum half-life of IgG, since IgG that is not bound by FcRn enters the lysosomal pathway and is degraded.

During the first stages of life, FcRn confers passive immunity to offspring before and after birth by mediating transfer of IgG across the maternal placenta or neonatal intestinal walls. FcRn continues to function throughout adult life and is expressed in various tissues, e.g., the epithelium of the lung and liver, the vascular endothelium, as well as in monocytes, macrophages, and dendritic cells.

FcRn-deficient mice are more resistant to autoimmune diseases caused by pathogenic IgG autoantibodies because they are unable to maintain high concentrations of pathogenic serum IgG. Accordingly, specific blockade of FcRn-IgG interactions can be used to promote degradation of pathogenic IgG antibodies, for example to treat IgG-mediated autoimmune diseases. FcRn also plays a critical role in major histocompatibility complex (MHC) class II antigen presentation and MHC class I cross-presentation of IgG-complexed antigen. When antigen is presented as an IgG-containing immune complex (IC), dendritic cells that are CD8⁻CD11b⁺CD11c⁺ (inflammatory dendritic cells) display significant cross-presentation at low antigen doses in a pathway that is highly dependent upon FcRn expression. This pathway involves the internalization of the ICs by Fcγ receptors into an acidic endosome. Subsequent binding of the ICs by FcRn within antigen presenting cells (APCs) initiates specific mechanisms that result in trafficking of the antigen-bearing IC into compartments where antigen is processed into peptide epitopes compatible with loading onto MHC. Thus, FcRn in dendritic cells enhances MHC II antigen presentation and induces proliferation of antigen-specific CD4⁺ T-cells as well as exhibits a fundamental role in antigen presentation to CD8⁺ T cells (cytotoxic T cells). This latter CD8⁺ T cell-pathway is called cross-presentation and involves the crossover of extracellular antigens into an MHC class I-dependent pathway. Blockade of FcRn-Ig IC interaction inhibits antigen presentation of IC and subsequent T cell activation stimulated by immune-associated antigen presentation. Interactions with IgG IC in APCs such as dendritic cells also promote secretion of inflammatory cytokines such as IL-12, IFNγ, and TNFα. Thus, blockade of FcRn-Ig IC interaction is useful to inhibit production of inflammatory cytokines by innate immune cells and antigen-activated T cells.

Pemphigus is a rare group of blistering autoimmune diseases that affect the skin and mucous membranes. The pathogenesis of pemphigus, including, but not limited to pemphigus vulgaris and pemphigus foliaceus, is related to the binding of IgG autoantibodies to keratinocyte antigens. The primary antigenic targets of pathogenic autoantibodies are desmoglein 1 and 3 (Dsg 1 and Dsg 3), cadherin family proteins that partially comprise the desmosome, a protein structure responsible for maintaining cell adhesion. IgG autoantibody binding to Dsg leads to a loss of epidermal keratinocyte adhesion, which in turn causes intra-epidermal blistering and the clinical appearance of flaccid blisters and erosions. Blockade of FcRn decreases total IgG levels in pemphigus patients, including a corresponding decrease in the levels of the pathogenic autoantibodies. This can lead to a decrease in the mucosal and cutaneous manifestations in patients with pemphigus, including, but not limited to pemphigus vulgaris and pemphigus foliaceus.

Pemphigoid diseases are characterized by the presence of autoantibodies against distinct structural components of the dermal-epidermal junction. Junction proteins link the cytoskeleton of the basal keratinocytes to the extracellular matrix of the dermis, and binding of pemphigoid autoantibodies leads to the separation of the epidermis. The pathogenesis of many pemphigoid diseases, including, but not limited to bullous pemphigoid and mucous membrane pemphigoid, is related to the binding of IgG autoantibodies to antigens including, but not limited to, laminin 332, and/or hemidesmosomal proteins BP180 or BP230. As noted above, blockade of FcRn decreases total IgG levels, including a corresponding decrease in the levels of the pathogenic autoantibodies, which benefits patients with pemphigoid diseases mediated by IgG autoantibodies.

In one aspect, provided are methods for treating pemphigus and/or a pemphigoid disease by administering to a subject in need thereof a therapeutically effective amount of an FcRn inhibitor (e.g., an antibody or antigen-binding fragment thereof that specifically binds FcRn, or any other “FcRn inhibitor” as described herein). Herein, references to anti-FcRn antibodies in particular are provided to illustrate a representative FcRn inhibitor, and do not limit the scope of the invention.

As used herein, the terms “treating”, “treat”, or the like, mean to alleviate or reduce the severity of at least one symptom or indication, to eliminate the causation of symptoms either on a temporary or permanent basis, or to obtain beneficial or desired clinical results. Beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment. Symptoms of pemphigus that may be lessened or eliminated by the methods disclosed herein include, but are not limited to, fluid-filled skin blisters, ruptured blisters, scaly, inflamed, painful patches on the skin, burning, pain, and itching at the site of the blisters, and/or chronic skin infections due to ruptured and irritated blisters. Symptoms of pemphigoid diseases that may be lessened or eliminated by the methods disclosed herein include, but are not limited to, fluid-filled skin blisters, ruptured blisters, itching skin, eczema and a hive-like rash. When the mucous membranes of the mouth are affected, symptoms can further include pain, burning, peeling away of affected inner lining tissues, and sensitivity to acidic foods.

In the methods described herein, a therapeutically effective amount of an FcRn inhibitor is administered to a subject in need thereof. By “subject” is meant a mammal, including, but not limited to, a human or non-human mammal, such as a bovine, equine, canine, ovine, or feline, etc. Individuals and patients are also subjects herein. “Therapeutically effective amount” means an amount of FcRn inhibitor set forth herein that, when administered to a mammal, is effective in producing a therapeutic effect.

In some embodiments, the pemphigus is pemphigus vulgaris, pemphigus foliaceus, paraneoplastic pemphigus, drug-induced pemphigus, endemic pemphigus (fogo selvagem), pemphigus erythematosus (Senear-Usher syndrome), or pemphigus vegetans.

In some embodiments, the pemphigoid disease is bullous pemphigoid, mucous membrane pemphigoid, pemphigoid gestationis, epidermolysis bullosa acquisita, anti-laminin g1/anti-p200 pemphigoid, or lichen planus pemphigoid.

In one aspect, administration of the FcRn inhibitor promotes degradation of pathogenic IgG antibodies in monomeric form. In another aspect, administration of the FcRn inhibitor promotes degradation of pathogenic IgG antibodies that are present as IgG-containing immune complexes (IC).

In one aspect, provided is a method of reducing total IgG levels in a subject in need thereof, the method comprising selecting a subject with pemphigus and/or a pemphigoid disease and administering to the subject one or more doses of a therapeutically effective amount of an FcRn inhibitor. In some embodiments, the total IgG level is decreased by about 5%, about 10%, about 15%, about 25%, about 30%, about 35%, about 40%, about 45%, about 50%, about 55%, about 60%, about 65%, about 70%, about 75%, about 80%, about 85%, about 90%, about 95%, about 100% as compared to a control level. A “control level” can refer to a level measured in one or more samples derived from one or more individuals that suffer from or have been diagnosed with pemphigus and/or a pemphigoid disease. The level may be measured on an individual-by-individual basis, or on an aggregate basis such as an average. In some embodiments, the control level is measured for the same individual whose condition is being monitored, but is obtained at a different time. In certain embodiments, a “control” level can refer to a level obtained from the same patient at an earlier time, e.g., weeks, months, or years earlier. In some embodiment, the control level is obtained from a patient before the patient received any therapy for pemphigus and/or a pemphigoid disease.

In one aspect, provided is a method of reducing circulating immune complex (CIC) levels in a subject in need thereof, the method comprising selecting a subject with pemphigus and/or a pemphigoid disease and administering to the subject one or more doses of a therapeutically effective amount of an FcRn inhibitor. In some embodiments, the CIC level is decreased by about 5%, about 10%, about 15%, about 25%, about 30%, about 35%, about 40%, about 45%, about 50%, about 55%, about 60%, about 65%, about 70%, about 75%, about 80%, about 85%, about 90%, about 95%, about 100% as compared to a control level.

In another aspect, provided is a method of reducing anti-Dsg 1 and/or anti-Dsg 3 antibody levels in a subject in need thereof, the method comprising selecting a subject with pemphigus and/or a pemphigoid disease and administering to the subject one or more doses of a therapeutically effective amount of an FcRn inhibitor. In some embodiments, the anti-Dsg 1 level and/or anti-Dsg 3 antibody level is decreased by about 5%, about 10%, about 15% , about 25%, about 30%, about 35%, about 40%, about 45%, about 50%, about 55%, about 60%, about 65%, about 70%, about 75%, about 80%, about 85%, about 90%, about 95%, about 100% as compared to a control level.

The methods disclosed herein include administering a therapeutically effective amount of an FcRn inhibitor. As used herein, an “FcRn inhibitor” refers to any molecule capable of inhibiting, blocking, abrogating or interfering with the interactions between FcRn and IgG. In some embodiments, the FcRn inhibitor can be an antibody or antigen-binding fragment thereof, a small molecule compound, a nucleic acid, a polypeptide, or a functional fragment or variant thereof. Other non-limiting examples of suitable FcRn inhibitors include RNAi molecules such as anti-FcRn RNAi molecules, antisense molecules such as anti-FcRn antisense RNA, and dominant negative proteins such as a dominant negative FcRn protein.

As used herein, the term “antibody” refers to an immunoglobulin molecule comprising four polypeptide chains, two heavy (H) chains and two light (L) chains inter-connected by disulfide bonds, as well as multimers thereof (e.g., IgM). In a typical antibody, each heavy chain comprises a heavy chain variable region (abbreviated herein as HCVR or VH) and a heavy chain constant region. The heavy chain constant region comprises three domains, CHL CH2 and CH3. Each light chain comprises a light chain variable region (abbreviated herein as LCVR or VL) and a light chain constant region. The light chain constant region comprises one domain (CL1). The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. In different embodiments of the invention, the FRs of the anti-FcRn antibody (or antigen-binding portion thereof) may be identical to the human germline sequences, or may be naturally or artificially modified. An amino acid consensus sequence may be defined based on a side-by-side analysis of two or more CDRs.

As used herein, the terms “antigen-binding portion” of an antibody, “antigen-binding fragment” of an antibody, and the like, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable immunoglobulin new antigen receipt (IgNAR) domains, are also encompassed within the expression “antigen-binding fragment,” as used herein.

An antigen-binding fragment will typically comprise at least one variable domain. The variable domain may be of any size or amino acid composition and will generally comprise at least one CDR which is adjacent to or in frame with one or more framework sequences. In antigen-binding fragments having a VH domain associated with a VL domain, the VH and VL domains may be situated relative to one another in any suitable arrangement. For example, the variable region may be dimeric and contain VH-VH, VH-VL or VL-VL dimers. Alternatively, the antigen-binding fragment may contain a monomeric VH or VL domain.

In certain embodiments, an antigen-binding fragment may contain at least one variable domain covalently linked to at least one constant domain. Non-limiting, exemplary configurations of variable and constant domains that may be found within an antigen-binding fragment disclosed herein include: (i) VH-CH1; (ii) VH-CH2; (iii) VH-CH3; (iv) VH-CH1-CH2; (v) VH-CH1-CH2-CH3; (vi) VH-CH2-CH3; (vii) VH-CL; (viii) VL-CH1; (ix) VL-CH2; (x) VL-CH3; (xi) VL-CH1-CH2; (xii) VL-CH1-CH2-CH3; (xiii) VL-CH2-CH3; and (xiv) VL-CL. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody provided herein may comprise a homo-dimer or hetero-dimer (or other multimer) of any of the variable and constant domain configurations listed above in non-covalent association with one another and/or with one or more monomeric VH or VL domain (e.g., by disulfide bond(s)).

In some embodiments, the methods disclosed herein comprise administering an anti-FcRn antibody or antigen-binding fragment thereof, wherein the anti-FcRn antibody is a chimeric, humanized, or human antibody.

As used herein, a “chimeric antibody” refers to a polypeptide comprising at least the antigen-binding portion of an and body molecule linked to at least part of another protein (typically an immunoglobulin constant domain derived from a human antibody).

As used herein, a “humanized antibody” refers to an antibody with a framework region (FR) having substantially the amino acid sequence of a human immunoglobulin and a complementarity determining region (CDR) having substantially the amino acid sequence of a non-human immunoglobulin (the “import” sequences). In certain embodiments, humanization of an antibody can reduce immunogenicity. In certain embodiments, the frameworks of the humanized antibody are a composite of two or more human antibodies. In other embodiments, surface-exposed framework residues of the antibody are replaced with framework residues of a human antibody to form a humanized antibody. In a preferred embodiment, the frameworks are selected to minimize the presence of amino acid sequences predicted to be. T cell epitopes over a wide population range.

As used herein, the term “human antibody” refers to an antibody having variable and constant regions derived from human germline immunoglobulin sequences. The human antibodies provided herein may nonetheless include amino acid residues not encoded by human germline immunoglobulin sequences (e.g., mutations introduced by random or site-specific mutagenesis in vitro or by somatic mutation in vivo), for example in the CDRs and in particular CDR3.

Anti-FcRn antibodies suitable for use in the methods disclosed herein further include those for which binding characteristics have been improved by direct mutation, methods of affinity maturation, phage display, or chain shuffling. Affinity and specificity can be modified or improved by mutating CDRs and screening for antigen binding sites having the desired characteristics (see, e.g., Yang et al., J. Mol. Biol., 254: 392-403 (1995)). CDRs can be mutated in a variety of ways. One way is to randomize individual residues or combinations of residues so that in a population of otherwise identical antigen binding sites, all twenty amino acids are found at particular positions. Alternatively, mutations may be induced over a range of CDR residues by error prone PCR methods (see, e.g., Hawkins et al., J. Mol. Biol., 226: 889-896 (1992)). For example, phage display vectors containing heavy and light chain variable region genes can be propagated in mutator strains of E. coli (see, e.g., Low et al., J. Mol. Biol., 250: 359-368 (1996)). These methods of mutagenesis are illustrative of the many methods known to one of skill in the art.

In some embodiments, the anti-FcRn antibodies or antigen-binding fragments thereof used in the methods disclosed herein may be obtained directly from hybridomas, which express the anti-FcRn antibodies or antigen-binding fragments thereof. In other embodiments, the anti-FcRn antibodies or antigen-binding fragments thereof may be cloned and recombinantly expressed in suitable host cells (e.g., CHO cells, NS/0 cells, HEK293 cells) Suitable host cells include plant cells, mammalian cells, and microorganisms such as E. coli and yeast. Alternatively, the anti-FcRn antibodies or antigen-binding fragments thereof may be produced recombinantly in a transgenic non-human animal or plant, e.g., a transgenic mouse.

In some embodiments, FcRn inhibitors used in the methods disclosed herein are antibodies or antigen-binding fragments thereof that specifically bind FcRn via the variable region of the anti-FcRn antibody or antigen-binding fragment thereof. The term “specifically binds,” or the like, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. For example, an antibody that “specifically binds” FcRn includes antibodies that bind FcRn or a portion thereof with a dissociation constant (KD) of less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 4 nM, less than about 3 nM, less than about 2 nM, less than about 1 nM or less than about 0.5 nM, as measured in a surface plasmon resonance assay. An isolated antibody that specifically binds human FcRn may, however, have cross-reactivity to other antigens, such as FcRn molecules from other (non-human) species.

According to certain exemplary embodiments, the FcRn antibody or antigen-binding fragment thereof comprises a heavy chain variable region (HCVR), light chain variable region (LCVR), and/or complementarity determining regions (CDRs) comprising the amino acid sequences of any of the anti-FcRn antibodies or antigen-binding fragments thereof set forth in US Patent Application Publication No. US2018/0291101, which is hereby incorporated by reference in its entirety.

In certain exemplary embodiments, the anti-FcRn antibody or antigen-binding fragment thereof comprises a heavy chain variable region comprising the amino acid sequence of SEQ ID NO: 1 and a light chain variable region comprising the amino acid sequence of SEQ ID NO: 2.

According to certain embodiments, the anti-FcRn antibody or antigen-binding fragment thereof comprises three heavy chain CDRs (HCDR1, HCDR2 and HCDR3) and three light chain CDRs (LCDR1, LCDR2 and LCDR3), wherein HCDR1 comprises the amino acid sequence of SEQ ID NO: 3; HCDR2 comprises the amino acid sequence of SEQ ID NO: 4; HCDR3 comprises the amino acid sequence of SEQ ID NO: 5; LCDR1 comprises the amino acid sequence of SEQ ID NO: 6; LCDR2 comprises the amino acid sequence of SEQ ID NO: 7; and LCDR3 comprises the amino acid sequence of SEQ ID NO: 8.

In certain embodiments, the methods provided herein comprise the use of an anti-FcRn antibody, wherein the antibody comprises a heavy chain comprising the amino acid sequence of SEQ ID NO: 9. In some embodiments, the anti-FcRn antibody comprises a light chain comprising the amino acid sequence of SEQ ID NO: 10.

An exemplary antibody comprising a heavy chain comprising the amino acid sequence of SEQ ID NO:9 and a light chain comprising the amino acid sequence of SEQ ID NO: 10 is a humanized, affinity-matured IgG4-K monoclonal antibody (mAb) that blocks IgG and IC interactions with FcRn, and inhibits the varied roles of FcRn in the immune response.

According to certain exemplary embodiments, the methods provided herein comprise the use of this antibody, or a bioequivalent thereof. As used herein, the term “bioequivalent” refers to anti-FcRn antibodies or FcRn-binding proteins or fragments thereof that are pharmaceutical equivalents or pharmaceutical alternatives whose rate and/or extent of absorption do not show a significant difference with that of a reference antibody (e.g., the antibody comprising a heavy chain comprising the amino acid sequence of SEQ ID NO: 9 and a light chain comprising the amino acid sequence of SEQ ID NO: 10) when administered at the same molar dose under similar experimental conditions, either single dose or multiple dose. The term “bioequivalent” includes antigen-binding proteins that bind to FcRn and do not have clinically meaningful differences from this reference antibody with respect to safety, purity and/or potency.

In some embodiments, the anti-FcRn antibody or antigen-binding fragment thereof comprises a heavy chain variable region having at least 80%, at least 90%, at least 95%, at least 98%, or at least 99% sequence identity to SEQ ID NO: 1.

In some embodiments, the anti-FcRn antibody or antigen-binding fragment thereof comprises a light chain variable region having at least 80%, at least 90%, at least 95%, at least 98%, or at least 99% sequence identity to SEQ ID NO: 2.

Sequence identity may be measured by methods known in the art (e.g., GAP, BESTFIT, and BLAST).

Also provided is the use of anti-FcRn antibodies or antigen-binding fragments thereof to treat pemphigus and/or a pemphigoid disease, wherein the anti-FcRn antibodies or antigen-binding fragments thereof comprise variants of any of the heavy or light chain variable regions and/or CDR amino acid sequences disclosed herein having one or more conservative amino acid substitutions. For example, provided is the use of anti-FcRn antibodies or antigen-binding fragments thereof having heavy or light chain variable regions and/or CDR amino acid sequences with, e.g., 10 or fewer, 8 or fewer, 6 or fewer, 4 or fewer, etc. conservative amino acid substitutions relative to any of the heavy or light chain variable regions and/or CDR amino acid sequences disclosed herein.

Other anti-FcRn antibodies or antigen-binding fragments thereof that can be used in the context of the methods provided herein include, but are not limited to, anti-FcRn antibodies DX-2500, DX-2504, DX-2507, HL161, Rozanolixizumab (UCB7665), and M281. Additional FcRn inhibitors that can be used in the context of the methods provided herein include FcRn inhibitors (including anti-FcRn antibodies) described in Patent Corporation Treaty applications PCT/US2009/002536, PCT/US2012/040409, PCT/KR2014/005495, PCT/KR2015/004424, PCT/EP2013/059802, PCT/EP2014/074409, PCT/US2016/015720, or PCT/US2017/044765, or in U.S. Pat. No. 7,662,928. The portions of all of the aforementioned publications that identify FcRn inhibitors, anti-FcRn antibodies and antigen-binding fragments thereof are hereby incorporated by reference.

In some embodiments, the FcRn inhibitor is an anti-FcRn antibody or antigen-binding fragment thereof comprising three heavy chain CDRs (HCDR1, HCDR2 and HCDR3) and three light chain CDRs (LCDR1, LCDR2 and LCDR3), wherein HCDR1 comprises the amino acid sequence EYAMG (SEQ ID NO: 11) or VYAMG (SEQ ID NO: 12); HCDR2 comprises the amino acid sequence SIGSSGGQTKYADSVKG (SEQ ID NO: 13) or SIGSSGGPTKYADSVKG (SEQ ID NO: 14); HCDR3 comprises the amino acid sequence LSTGELY (SEQ ID NO: 15), LSIRELV (SEQ ID NO: 16), LSIVDSY (SEQ ID NO: 17), LSLGDSY (SEQ ID NO: 18), or LAIGDSY (SEQ ID NO: 19); LCDR1 comprises the amino acid sequence TGTGSDVGSYNLVS (SEQ ID NO: 20); LCDR2 comprises the amino acid sequence GDSQRPS (SEQ ID NO: 21); and LCDR3 comprises the amino acid sequence CSYAGSGIYV (SEQ ID NO: 22).

In some embodiments, the FcRn inhibitor is an anti-FcRn antibody or antigen-binding fragment thereof comprising three heavy chain CDRs (HCDR1, HCDR2 and HCDR3) and three light chain CDRs (LCDR1, LCDR2 and LCDR3), wherein HCDR1 comprises the amino acid sequence EYAMG (SEQ ID NO: 11); HCDR2 comprises the amino acid sequence SIGSSGGQTKYADSVKG (SEQ ID NO: 13); HCDR3 comprises the amino acid sequence LAIGDSY (SEQ ID NO: 19); LCDR1 comprises the amino acid sequence TGTGSDVGSYNLVS (SEQ ID NO: 20); LCDR2 comprises the amino acid sequence GDSQRPS (SEQ ID NO: 21); and LCDR3 comprises the amino acid sequence SSYAGSGIYV (SEQ ID NO: 23) or ASYAGSGIYV (SEQ ID NO: 24).

In some embodiments, the FcRn inhibitor is an anti-FcRn antibody or antigen-binding fragment thereof comprising three heavy chain CDRs (HCDR1, HCDR2 and HCDR3) and three light chain CDRs (LCDR1, LCDR2 and LCDR3), wherein HCDR1 comprises the amino acid sequence GFTFSNYGMV (SEQ ID NO: 25); HCDR2 comprises the amino acid sequence YIDSDGDNTYYRDSVKG (SEQ ID NO: 26); HCDR3 comprises the amino acid sequence GIVRPFLY (SEQ ID NO: 27); LCDR1 comprises the amino acid sequence KSSQSLVGASGKTYLY (SEQ ID NO: 28); LCDR2 comprises the amino acid sequence LVSTLDS (SEQ ID NO: 29); and LCDR3 comprises the amino acid sequence LQGTHFPHT (SEQ ID NO: 30).

In some embodiments, the FcRn inhibitor is an anti-FcRn antibody or antigen-binding fragment thereof comprising three heavy chain CDRs (HCDR1, HCDR2 and HCDR3) and three light chain CDRs (LCDR1, LCDR2 and LCDR3), wherein HCDR1 comprises the amino acid sequence GFSLSTYGVGVG (SEQ ID NO: 31); HCDR2 comprises the amino acid sequence NIWWDDDKRYNPSLEN (SEQ ID NO: 32); HCDR3 comprises the amino acid sequence TPAYYGSHPPFDY (SEQ ID NO: 33); LCDR1 comprises the amino acid sequence RTSEDIYTNLA (SEQ ID NO: 34); LCDR2 comprises the amino acid sequence VAKTLQD (SEQ ID NO: 35); and LCDR3 comprises the amino acid sequence LQGFKFPWT (SEQ ID NO: 36).

In some embodiments, the FcRn inhibitor is an anti-FcRn antibody or antigen-binding fragment thereof comprising three heavy chain CDRs (HCDR1, HCDR2 and HCDR3) and three light chain CDRs (LCDR1, LCDR2 and LCDR3), wherein HCDR1 comprises the amino acid sequence FSYWV (SEQ ID NO: 37); HCDR2 comprises the amino acid sequence TIYYSGNTYYNPSLKS (SEQ ID NO: 38); HCDR3 comprises the amino acid sequence RAGILTGYLDS (SEQ ID NO: 39); LCDR1 comprises the amino acid sequence GGNNIGSKSVH (SEQ ID NO: 40); LCDR2 comprises the amino acid sequence DDSDRPS (SEQ ID NO: 41); and LCDR3 comprises the amino acid sequence QVWDSSSDHVV (SEQ ID NO: 42).

In some embodiments, the FcRn inhibitor is an anti-FcRn antibody or antigen-binding fragment thereof comprising three heavy chain CDRs (HCDR1, HCDR2 and HCDR3) and three light chain CDRs (LCDR1, LCDR2 and LCDR3), wherein HCDR1 comprises the amino acid sequence TYAMG (SEQ ID NO: 43); HCDR2 comprises the amino acid sequence SIGASGSQTRYADS (SEQ ID NO: 44); HCDR3 comprises the amino acid sequence LAIGDSY (SEQ ID NO: 19); LCDR1 comprises the amino acid sequence TGTGSDVGSYNLVS (SEQ ID NO: 20); LCDR2 comprises the amino acid sequence GDSERPS (SEQ ID NO: 45); and LCDR3 comprises the amino acid sequence SSYAGSGIYV (SEQ ID NO: 23).

In other embodiments, FcRn inhibitors used in the methods disclosed herein are FcRn inhibitors that bind FcRn via an Fc region, or FcRn-binding fragment thereof In one embodiment, the FcRn inhibitor that binds FcRn via an Fc region, or FcRn-binding fragment thereof, comprises an antibody variable region and/or a CH1 domain. In one embodiment, the FcRn inhibitor that binds FcRn via an Fc region, or FcRn-binding fragment thereof, does not comprise an antibody variable region and/or a CH1 domain. In one embodiment, the FcRn inhibitor is an FcRn inhibitor described in PCT/EP2011/050071, PCT/US2014/072087 or PCT/M2016/000398. The portions of the aforementioned publications that identify FcRn inhibitors are hereby incorporated by reference. In one embodiment, the FcRn inhibitor comprises an Fc domain comprising SEQ ID NO:46, SEQ ID NO:47, or SEQ ID NO:48.

The methods of this disclosure may use any of the FcRn inhibitors, anti-FcRn antibodies or antigen-binding fragments thereof disclosed and/or incorporated by reference herein.

In the methods disclosed herein, therapeutic compositions comprising an FcRn inhibitor may be administered in any convenient manner, including by injection, infusion, transfusion, implantation or transplantation. The compositions used in the methods described herein may be administered to a patient subcutaneously, intradermally, intratumorally, intranodally, intramedullary, intramuscularly, intracranially, by intravenous or intralymphatic injection, by intravenous or intralymphatic infusion, or intraperitoneally. In one embodiment, the compositions used in the methods disclosed herein are preferably administered by intravenous infusion. In another embodiment, the compositions used in the methods disclosed herein are preferably administered by subcutaneous infusion or injection.

In certain embodiments, the FcRn inhibitor, such as an anti-FcRn antibody or antigen-binding fragment thereof, is administered to the mammal by intravenous infusion, i.e., introduction of the antibody or antigen-binding fragment thereof into the vein of a mammal over a certain period of time. In certain embodiments, the period of time is about 5 minutes, about 10 minutes, about 30 minutes, about 1 hour, about 2 hours, about 4 hours, or about 8 hours.

In certain embodiments, the methods disclosed herein include administering the FcRn inhibitor, such as an anti-FcRn antibody or antigen-binding fragment thereof, to the subject in need thereof in multiple doses, e.g., as part of a specific therapeutic dosing regimen. In some embodiments, the therapeutic dosing regimen may comprise administering one or more doses of the FcRn inhibitor to the subject at a frequency of once a week or once every other week.

In certain embodiments, the one or more doses are administered in at least one treatment cycle. A treatment cycle may comprise one or more initial, one or more secondary, and one or more tertiary doses. The methods, according to this aspect, comprise administering to a subject in need thereof at least one treatment cycle comprising administration of 3, 5, 8, or more doses of an FcRn inhibitor (such as an anti-FcRn antibody or antigen-binding fragment thereof). In one embodiment, a treatment cycle comprises 3 doses of an FcRn inhibitor. In one embodiment, a treatment cycle comprises 5 doses of an FcRn inhibitor. In one embodiment, a treatment cycle comprises 8 doses of an FcRn inhibitor.

The amount of an FcRn inhibitor contained within an individual dose may be expressed in terms of milligrams of antibody per kilogram of subject body weight (i.e., mg/kg). In certain embodiments, each dose of the FcRn inhibitor, such as an anti-FcRn antibody or antigen-binding fragment thereof, comprises 10 or 30 mg/kg of the patient's body weight. In one embodiment, each dose of the FcRn inhibitor comprises 10 mg/kg of the patient's body weight. In one embodiment, each dose of the FcRn inhibitor comprises 30 mg/kg of the patient's body weight.

In some embodiments, one or more initial doses are administered as loading doses. In some embodiments, the one or more initial loading doses are followed by one or more secondary doses administered as maintenance doses. In other embodiments, one or more initial and secondary doses are followed by one or more tertiary doses. The initial, secondary, and/or tertiary doses may all contain the same amount of the FcRn inhibitor, such as an anti-FcRn antibody or antigen-binding fragment thereof. In certain embodiments, however, the amount of the FcRn inhibitor contained in the initial, secondary and/or tertiary dose varies from one another (e.g., is higher or lower as appropriate).

In some embodiments, one or more initial doses comprising an anti-FcRn antibody or antigen-binding fragment thereof are administered to a patient with pemphigus and/or a pemphigoid disease, wherein the one or more initial doses comprise the anti-FcRn antibody or antigen-binding fragment thereof at 30 mg/kg of the subject's body weight. In some embodiments, the one or more initial doses are followed by one or more secondary doses, wherein the one or more secondary doses comprise the anti-FcRn antibody or antigen-binding fragment thereof at 10 mg/kg of the subject's body weight. In one embodiment, the one or more initial doses are administered once a week. In one embodiment, the one or more secondary doses are administered once every other week.

In one aspect, provided is a method of treating pemphigus and/or a pemphigoid disease in a subject in need thereof, the method comprising administering to the subject an anti-FcRn antibody or antigen-binding fragment thereof, wherein the FcRn antibody or antigen-binding fragment thereof is administered at a dose of 10 mg/kg of the subject's body weight once a week. In one embodiment, the anti-FcRn antibody antigen-binding fragment thereof is administered at a dose of 10 mg/kg once a week for at least five weeks. In one embodiment, the anti-FcRn antibody or antigen-binding fragment thereof is administered at a dose of 10 mg/kg once a week for five weeks. In one embodiment, the anti-FcRn antibody comprises a heavy chain comprising the amino acid sequence of SEQ ID NO: 9 and a light chain comprising the amino acid sequence of SEQ ID NO: 10. In one embodiment, the anti-FcRn antibody or antigen-binding fragment thereof comprises a heavy chain variable region comprising the amino acid sequence of SEQ ID NO: 1 and a light chain variable region comprising the amino acid sequence of SEQ ID NO: 2. In one embodiment, the anti-FcRn antibody or antigen-binding fragment thereof comprises three heavy chain CDRs (HCDR1, HCDR2 and HCDR3) and three light chain CDRs (LCDR1, LCDR2 and LCDR3), wherein HCDR1 comprises the amino acid sequence of SEQ ID NO: 3; HCDR2 comprises the amino acid sequence of SEQ ID NO: 4; HCDR3 comprises the amino acid sequence of SEQ ID NO: 5; LCDR1 comprises the amino acid sequence of SEQ ID NO: 6; LCDR2 comprises the amino acid sequence of SEQ ID NO: 7; and LCDR3 comprises the amino acid sequence of SEQ ID NO: 8.

In one aspect, provided is a method of treating pemphigus and/or a pemphigoid disease in a subject in need thereof, the method comprising administering to the subject an initial dose of an anti-FcRn antibody or antigen-binding fragment thereof, wherein the initial dose comprises the anti-FcRn antibody or antigen-binding fragment thereof at 30 mg/kg of the subject's body weight. In some embodiments, the initial dose is administered once a week. In one embodiment, the anti-FcRn antibody or antigen-binding fragment thereof is administered at an initial dose of 30 mg/kg of the subject's body weight once a week for at least three weeks. In one embodiment, the anti-FcRn antibody or antigen-binding fragment thereof is administered at an initial dose of 30 mg/kg of the subject's body weight once a week for three weeks. In certain embodiments, the method further comprises administering to the subject a secondary dose of the anti-FcRn antibody or antigen-binding fragment thereof, wherein the secondary dose comprises the anti-FcRn antibody or antigen-binding fragment thereof at 10 mg/kg of the subject's body weight. In one embodiment, the secondary dose is administered every other week. In one embodiment, the secondary dose is administered every other week for at least five weeks. In one embodiment, the secondary dose is administered every other week for five weeks. In one embodiment, the anti-FcRn antibody comprises a heavy chain comprising the amino acid sequence of SEQ ID NO: 9 and a light chain comprising the amino acid sequence of SEQ ID NO: 10. In one embodiment, the anti-FcRn antibody or antigen-binding fragment thereof comprises a heavy chain variable region comprising the amino acid sequence of SEQ ID NO: 1 and a light chain variable region comprising the amino acid sequence of SEQ ID NO: 2. In one embodiment, the anti-FcRn antibody or antigen-binding fragment thereof comprises three heavy chain CDRs (HCDR1, HCDR2 and HCDR3) and three light chain CDRs (LCDR1, LCDR2 and LCDR3), wherein HCDR1 comprises the amino acid sequence of SEQ ID NO: 3; HCDR2 comprises the amino acid sequence of SEQ ID NO: 4; HCDR3 comprises the amino acid sequence of SEQ ID NO: 5; LCDR1 comprises the amino acid sequence of SEQ ID NO: 6; LCDR2 comprises the amino acid sequence of SEQ ID NO: 7; and LCDR3 comprises the amino acid sequence of SEQ ID NO: 8.

In certain embodiments, each dose comprises 100-4500 mg of the FcRn antibody or antigen-binding fragment thereof, for example 100, 500, 1,000, 1,500, 2,000, 2,500, 3,000, 3,500, 4,000, 4,500 mg or more of the anti-FcRn antibody or antigen-binding fragment thereof. In one embodiment, the dose comprises 10 mg/kg of an anti-FcRn antibody or antigen-binding fragment thereof. In one embodiment, the dose comprises 30 mg/kg of an anti-FcRn antibody or antigen-binding fragment thereof. In one embodiment, the anti-FcRn antibody comprises a heavy chain comprising the amino acid sequence of SEQ ID NO: 9 and a light chain comprising the amino acid sequence of SEQ ID NO: 10. In one embodiment, the anti-FcRn antibody or antigen-binding fragment thereof comprises a heavy chain variable region comprising the amino acid sequence of SEQ ID NO: 1 and a light chain variable region comprising the amino acid sequence of SEQ ID NO: 2. In one embodiment, the anti-FcRn antibody or antigen-binding fragment thereof comprises three heavy chain CDRs (HCDR1, HCDR2 and HCDR3) and three light chain CDRs (LCDR1, LCDR2 and LCDR3), wherein HCDR1 comprises the amino acid sequence of SEQ ID NO: 3; HCDR2 comprises the amino acid sequence of SEQ ID NO: 4; HCDR3 comprises the amino acid sequence of SEQ ID NO: 5; LCDR1 comprises the amino acid sequence of SEQ ID NO: 6; LCDR2 comprises the amino acid sequence of SEQ ID NO: 7; and LCDR3 comprises the amino acid sequence of SEQ ID NO: 8.

In one embodiment, the FcRn inhibitor can be formulated with one or more pharmaceutically acceptable excipients.

The pharmaceutical compositions used in the methods disclosed herein may be specially formulated in solid or liquid form, including those adapted for parenteral administration, for example, by subcutaneous, intratumoral, intramuscular or intravenous injection or infusion as, for example, a sterile solution or suspension.

Injectable formulations or formulations for infusion of the pharmaceutical compositions used in the methods disclosed herein may be prepared by known methods. The injectable or infusible formulation thus prepared is preferably filled in an appropriate injection ampoule or in a vial or bag suitable for infusion.

A pharmaceutically acceptable excipient can be a pharmaceutically acceptable material, composition or vehicle, such as a liquid or solid filler, diluent, carrier, manufacturing aid (e.g., lubricant, talc magnesium, calcium or zinc stearate, or steric acid), solvent or encapsulating material involved in carrying or transporting the therapeutic compound for administration to the subject, bulking agent, salt, surfactant and/or a preservative. Some examples of materials which can serve as pharmaceutically acceptable excipients include: sugars, such as lactose, glucose and sucrose; starches, such as corn starch and potato starch; cellulose and its derivatives, such as sodium carboxymethyl cellulose, ethyl cellulose and cellulose acetate; gelatin; talc; waxes; oils, such as peanut oil, cottonseed oil, safflower oil, sesame oil, olive oil, corn oil and soybean oil; glycols, such as ethylene glycol and propylene glycol; polyols, such as glycerin, sorbitol, mannitol and polyethylene glycol; esters, such as ethyl oleate and ethyl laurate; agar; buffering agents; water; isotonic saline; pH buffered solutions; bulking agents such as mannitol, glycine, polyethylene glycol and sorbitol; surfactants such as polysorbates, poloxamers, Triton, sodium dodecyl sulfate (SDS), sodium laurel sulfate, polyethyl glycol, polypropyl glycol and copolymers of ethylene and propylene glycol; preservatives such as octadecyldimethylbenzyl ammonium chloride, hexamethonium chloride, benzalkonium chloride, benzethonium chloride, aromatic alcohols such as phenol, butyl and benzyl alcohol, alkyl parabens such as methyl or propyl paraben, catechol, resorcinol, cyclohexanol, 3-pentanol, and m-cresol, and other non-toxic compatible substances employed in pharmaceutical formulations.

Other suitable excipients can be found in standard pharmaceutical texts, e.g. in “Remington's Pharmaceutical Sciences”, The Science and Practice of Pharmacy, 19th Ed. Mack Publishing Company, Easton, Pa., (1995).

In some embodiments, the compositions comprising the FcRn inhibitor and the pharmaceutically acceptable carrier used in the methods disclosed herein are lyophilized and provided in a composition for reconstitution prior to administration.

The amino acid sequences cited in this application are listed in Table 1.

TABLE 1
Amino Acid Sequences

| SEQ | | |
|---|---|---|
| ID | | |
| NO: | Description Sequence | |
| 1 | Heavy chain | QVQLVQSGAELKKPGASVKLSCKASGYTFTSYGISWVKQATG |
| | variable | QGLEWIGEIYPRSGNTYYNEKFKGRATLTADKSTSTAYMELRS |
| | region | LRSEDSAVYFCARSTTVRPPGIWGTGTTVTVSS |
| 2 | Light chain | DIQMTQSPSSLSASVGDRVTITCKASDHINNWLAWYQQKPGQ |
| | variable | APRLLISGATSLETGVPSRFSGSGTGKDYTLTISSLQPEDFATYY |
| | region | CQQYWSTPYTFGGGTKVEIK |
| 3 | HCDR1 | SYGIS |
| 4 | HCDR2 | EIYPRSGNTYYNEKFKG |
| 5 | HCDR3 | STTVRPPGI |
| 6 | LCDR1 | KASDHINNWLA |
| 7 | LCDR2 | GATSLET |
| 8 | LCDR3 | QQYWSTPYT |
| 9 | Heavy chain | QVQLVQSGAELKKPGASVKLSCKASGYTFTSYGISWVKQATG |
| | (without | QGLEWIGEIYPRSGNTYYNEKFKGRATLTADKSTSTAYMELRS |
| | leader | LRSEDSAVYFCARSTTVRPPGIWGTGTTVTVSSASTKGPSVFPL |
| | sequence) | APCSRSTSESTAALGCLVKDYFPEPVTVSWNSGALTSGVHTFP |
| | | AVLQSSGLYSLSSVVTVPSSSLGTKTYTCNVDHKPSNTKVDKR |
| | | VESKYGPPCPPCPAPEFLGGPSVFLFPPKPKDTLMISRTPEVTC |
| | | VVVDVSQEDPEVQFNWYVDGVEVHNAKTKPREEQFNSTYRV |
| | | VSVLTVLHQDWLNGKEYKCKVSNKGLPSSIEKTISKAKGQPR |
| | | EPQVYTLPPSQEEMTKNQVSLTCLVKGFYPSDIAVEWESNGQP |
| | | ENNYKTTPPVLDSDGSFFLYSRLTVDKSRWQEGNVFSCSVMH |
| | | EALHNHYTQKSLSLSLG |
| 10 | Light chain | DIQMTQSPSSLSASVGDRVTITCKASDHINNWLAWYQQKPGQ |
| | (without | APRLLISGATSLETGVPSRFSGSGTGKDYTLTISSLQPEDFATYY |
| | leader | CQQYWSTPYTFGGGTKVEIKRTVAAPSVFIFPPSDEQLKSGTA |
| | sequence) | SVVCLLNNFYPREAKVQWKVDNALQSGNSQESVTEQDSKDS |
| | | TYSLSSTLTLSKADYEKHKVYACEVTHQGLSSPVTKSFNRGEC |
| 11 | HCDR1 | EYAMG |
| 12 | HCDR1 | VYAMG |
| 13 | HCDR2 | SIGSSGGQTKYADSVKG |
| 14 | HCDR2 | SIGSSGGPTKYADSVKG |
| 15 | HCDR3 | LSTGELY, |
| 16 | HCDR3 | LSIRELV, |
| 17 | HCDR3 | LSIVDSY, |
| 18 | HCDR3 | LSLGDSY |
| 19 | HCDR3 | LAIGDSY |
| 20 | LCDR1 | TGTGSDVGSYNLVS |
| 21 | LCDR2 | GDSQRPS |
| 22 | LCDR3 | CSYAGSGIYV |
| 23 | LCDR3 | SSYAGSGIYV |
| 24 | LCDR3 | ASYAGSGIYV |
| 25 | HCDR1 | GFTFSNYGMV |
| 26 | HCDR2 | YIDSDGDNTYYRDSVKG |
| 27 | HCDR3 | GIVRPFLY |
| 28 | LCDR1 | KSSQSLVGASGKTYLY |
| 29 | LCDR2 | LVSTLDS |
| 30 | LCDR3 | LQGTHFPHT |
| 31 | HCDR1 | GFSLSTYGVGVG |
| 32 | HCDR2 | NIWWDDDKRYNPSLEN |
| 33 | HCDR3 | TPAYYGSHPPFDY |
| 34 | LCDR1 | RTSEDIYTNLA |
| 35 | LCDR2 | VAKTLQD |
| 36 | LCDR3 | LQGFKFPWT |
| 37 | HCDR1 | FSYWV |
| 38 | HCDR2 | TIYYSGNTYYNPSLKS |
| 39 | HCDR3 | RAGILTGYLDS |
| 40 | LCDR1 | GGNNIGSKSVH |
| 41 | LCDR2 | DDSDRPS |
| 42 | LCDR3 | QVWDSSSDHVV |
| 43 | HCDR1 | TYAMG |
| 44 | HCDR2 | SIGASGSQTRYADS |
| 45 | LCDR2 | GDSERPS |
| 46 | Fc domain | CPPCPAPELLGGPSVFLFPPKPKDTLYITREPEVTCVVVDVSHE |
| | | DPEVKFNWYVDGVEVHNAKTKPREEQYNSTYRVVSVLTVLH |
| | | QDWLNGKEYKCKVSNKALPAPIEKTISKAKGQPREPQVYTLPP |
| | | SRDELTKNQVSLTCLVKGFYPSDIAVEWESNGQPENNYKTTPP |
| | | VLDSDGSFFLYSKLTVDKSRWQQGNVFSCSVMHEALKFHYTQ |
| | | KSLSLSPG |
| 47 | Fc domain | DKTHTCPPCPAPELLGGPSVFLFPPKPKDTLYITREPEVTCVVV |
| | | DVSHEDPEVKFNWYVDGVEVHNAKTKPREEQYNSTYRVVSV |
| | | LTVLHQDWLNGKEYKCKVSNKALPAPIEKTISKAKGQPREPQ |
| | | VYTLPPSRDELTKNQVSLTCLVKGFYPSDIAVEWESNGQPENN |
| | | YKTTPPVLDSDGSFFLYSKLTVDKSRWQQGNVFSCSVMHEAL |
| | | KFHYTQKSLSLSPGK |
| 48 | Fc domain | DKTHTCPPCPAPELLGGPSVFLFPPKPKDTLYITREPEVTCVVV |
| | | DVSHEDPEVKFNWYVDGVEVHNAKTKPREEQYNSTYRVVSV |
| | | LTVLHQDWLNGKEYKCKVSNKALPAPIEKTISKAKGQPREPQ |
| | | VYTLPPSRDELTKNQVSLTCLVKGFYPSDIAVEWESNGQPENN |
| | | YKTTPPVLDSDGSFFLYSKLTVDKSRWQQGNVFSCSVMHEAL |
| | | KFHYTQKSLSLSPG |

All referenced publications are incorporated herein by reference in their entirety. Furthermore, where a definition or use of a term in a reference, which is incorporated by reference herein, is inconsistent with or contrary to the definition of that term provided herein, the definition of that term provided herein applies and the definition of that term in the reference does not apply.

To facilitate a better understanding of the present invention, the following examples of specific embodiments are given. The following examples should not be read to limit or define the entire scope of the invention.

### EXAMPLE

### Example 1: Clinical Trial of Anti-FcRn Antibody Administered to Patients with Pemphigus

This study is a phase 1b/2, multicenter, open-label clinical trial, in which an anti-FcRn antibody is administered to patients with pemphigus.

The exemplary anti-FcRn antibody used in this study is a human monoclonal anti-FcRn antibody comprising a heavy chain comprising the amino acid sequence of SEQ ID NO: 9 and a light chain comprising the amino acid sequence of SEQ ID NO: 10; an HCVR/LCVR amino acid sequence pair comprising SEQ ID NOs: 1/2; and heavy and light chain CDR sequences comprising SEQ ID NOs: 3-8 (hereinafter referred to as the “study drug”).

Study Duration

The duration of subject participation for each cohort is summarized in Table 2.

TABLE 2
Duration of subject participation

| | | | | Maximum total | |
|---|---|---|---|---|---|
| Cohort | Screening | Treatment | Follow-up | Days | Weeks |
| 1 | ≤14 days | 28 days | 84 days | 126 days | 18 weeks |
| 2 | ≤14 days | 84 days | 56 days | 154 days | 22 weeks |

Study Population

Male or female subjects 18 years of age and older with a confirmed diagnosis of pemphigus (vulgaris or foliaceus).

Inclusion criteria—subjects must have met the following criteria to be eligible for the study: (1) willing and able to read, understand, and sign an informed consent form; (2) male or female ≥18 years of age at the time of screening; (3) documented diagnosis of pemphigus vulgaris or foliaceus based on all 3 of the following criteria: (a) documented clinical history consistent with pemphigus vulgaris or foliaceus (clinical presentation defined as mucosal and/or skin lesions), (b) presence of anti-Dsg 1 or 3 antibodies above the upper limit of normal (ULN), and (c) history of at least one positive tissue-based test (e.g., biopsy, direct immunofluorescence [DIF]); (4) Active disease defined as lesions lasting >2 weeks, and 3 active lesions in skin or mucosa or 2 active lesions with at least one being a skin lesion >1 cm diameter: (a) if treated with rituximab or other anti-CD20 mAb, last dose >9 months prior to screening, (b) if being treated with other immunosuppressants (i.e., azathioprine, mycophenolate mofetil, methotrexate, dapsone, cyclosporine, tacrolimus, sirolimus, or low-dose cyclophosphamide [≤100 mg/day]), dose must be stable, defined as <25% change in dose, for 4 weeks prior to screening, (c) on stable dose of corticosteroids, defined as ≤1 mg/kg of prednisone or equivalent and may not be increased by more than 50% in the 2 weeks prior to screening, (d) allowed topical therapies for pemphigus lesions upon entering the study include petroleum jelly or Aquaphor® for the skin or chlorhexidine for the mouth, (e) stable use of topical low strength hydrocortisone (≤1%), tacrolimus, sirolimus, or pimecrolimus for lesions contributing <10% of the Pemphigus Disease Area Index (PDAI) total activity score for the 4 weeks prior to screening is allowed; stable use of dexamethasone elixir solution (swish and spit only) for oral lesions for the 4 weeks prior to screening is allowed, (f) if not on regular corticosteroids, no pulse corticosteroids are allowed in the 2 weeks prior to screening; (5) body mass index (BMI) >18.5 kg/m2; (6) has a negative pregnancy test documented prior to the first dose of study drug (for women of childbearing potential); (7) females of childbearing potential must agree to be abstinent or else use any two of the following medically acceptable forms of contraception (<1% per year failure rate) from the screening period through the final study visit: oral contraceptive, condom with or without spermicidal jelly, diaphragm or cervical cap with spermicidal jelly, or intrauterine device (IUD); a female whose male partner has had a vasectomy must agree to use one additional form of medically acceptable contraception; (8) females of non-childbearing potential, defined as surgically sterile (status post hysterectomy, bilateral oophorectomy, or bilateral tubal ligation) or post-menopausal for at least 12 months do not require contraception during the study; (9) males with female partners of childbearing potential, including males who are surgically sterile (post vasectomy), must agree to be abstinent or else use a medically acceptable form of contraception from the screening period through the final study visit; and (10) a PDAI total activity score of >4 at screening.

Exclusion criteria—subjects meeting any of the following criteria were ineligible for the study: (1) subject unable or unwilling to comply with the protocol; (2) active non-hematologic malignancy or history of non-hematologic malignancy in the 3 years prior to screening (exclusive of non-melanoma skin cancer and cervical cancer in situ); (3) positive for human immunodeficiency virus (HIV) or hepatitis C antibody; (4) positive for hepatitis B surface antigen; (5) active infection or history of recurrent infections; (6) IVIG treatment within 30 days of screening; (7) received any cytotoxic (other than azathioprine) or any non-anti-CD20 mAb therapy in the 3 months prior to screening; (8) any exposure to an investigational drug or device within the 30 days prior to screening; (9) plasmapheresis or immunoadsorption within 30 days of screening; (10) cellular therapy, including chimeric antigen receptor and T-cell (CAR-T), at any time prior to screening; (11) participant had any current medical condition that may have compromised their safety or compliance, preclude successful conduct of the study, or interfere with interpretation of the results; (12) use of any systemic or topical immunosuppressive drugs within 3 months of screening not including dose allowed by the inclusion criteria; or (13) serum total IgG <600 mg/dL at Screening.

Study Variables

Primary endpoints in regards to safety include the determination of the study drug safety based on vital signs, physical examinations, electrocardiograms (ECGs), clinical safety laboratory tests, the incidence of adverse events (AEs), treatment-emergent adverse events (TEAEs) and serious adverse events (SAEs) summarized by dose and dosing regimen, severity, and relationship to study drug. Primary endpoints are the measurement of (i) a study drug-induced decrease in IgG levels nadir as compared to baseline and (ii) a study drug-induced reduction in the PDAI total activity score as compared to baseline.

Secondary endpoints for this study include: (1) the determination of pharmacodynamics (PD) biomarkers based on absolute serum levels and percent change from baseline of total IgG, IgG subtypes (IgG₁₋₄), immunoglobulin A (IgA), immunoglobulin M (IgM), albumin, CIC, anti-Dsg1 and anti-Dsg3 antibody titers, and complement component 3 (C3) and anti-epithelial cell antibody (AECA) levels by indirect immunofluorescence summarized by dose, dosing regimen and visit; (2) the determination of PK parameters including half-life (t_{1/2}), maximum serum concentration determined directly from the concentration-time profile (Cₘₐₓ), observed time of peak serum concentration (tₘₐₓ), area under the serum concentration-time curve from pre-dose (time₀) to 24 hours post-dose (AUC₀₋₂₄), and area under the serum concentration time curve from pre-dose (time₀) to infinity (AUC_{0-∞}); maximum serum concentration determined directly from the maximum serum concentration and corresponding tₘₐₓ summarized by dose, dosing regimen, visit and time point; (3) the assessment of pemphigus disease activity by responses on the PDAI based on absolute and percent change from baseline, summarized by dose, dosing regimen and visit; (4) the assessment of pemphigus severity and disease activity was using the PDAI; and (5) immunogenicity of the study drug as determined by presence of antibodies binding to the study drug and neutralizing antibodies summarized by dose, dosing regimen, visit and time point. An overview of the PK parameters is provided in Table 3.

Additional endpoints for this study include: (1) the mechanisms of action and effects of the study drug on pathophysiology summarized by dose, dosing regimen and visit as determined by (a) complement component 3 (C3) levels by nephelometry, (b) anti-epithelial cell antibody (AECA) titers by indirect immunofluorescence, (c) Fc gamma R2A receptor (FCGR2A) single nucleotide polymorphisms (SNP) by genotyping, (d) presence of disease and inflammatory markers by total RNA sequencing (RNAseq), (e) immunophenotyping including measurements of T cells, monocytes, natural killer (NK) cells and B cells by flow cytometry, (f) urine IgG levels to explore study drug distribution and elimination, and (g) exploratory biomarkers to determine immune response associated with pemphigus; (2) the evaluation of corticosteroid use during the study is to be summarized by dose, dosing regimen and visit; (3) the assessment of the impact of the study drug on subject's health-related quality of life (HR-QoL) by responses to the Autoimmune Bullous Diseases Quality of Life (ABQoL) questionnaire and Skindex-29 scores summarized by dose, dosing regimen and visit; (4) the qualitative assessment of changes in appearance of skin and mucosal lesions as determined by photography presented by dose, dosing regimen and visit; and (5) the determination of levels of the study drug in skin biopsies across time points (skin biopsies optional).

TABLE 3
PK parameters and description

| PK | |
|---|---|
| Parameter | Description |
| Cₘₐₓ | Maximum observed serum concentration observed |
| | directly from data |
| tₘₐₓ | Time to reach maximum observed concentration directly |
| | from data |
| λ_{z} | Apparent first-order terminal elimination rate constant |
| | calculated by linear regression of the terminal linear |
| | portion of the log concentration vs. time curve |
| t_{1/2} | Terminal elimination half-life, calculated as ln(2)/λ_{z} |
| AUC₀₋₂₄ | AUC from time zero to 24 hours postdose administration |
| AUC_{0-∞} | AUC from time zero to infinity time (as |
| | AUC₀₋ₜ + C_{last/λz}, where Cₗₐₛₜ is the last |
| | quantifiable concentration) |

Study Design

Up to 8 subjects with a diagnosis of pemphigus (vulgaris or foliaceus) received 10 mg/kg of the study drug weekly×5 doses (Cohort 1).

Up to 12 subjects with a diagnosis of pemphigus (vulgaris or foliaceus) received 30 mg/kg of the study drug weekly×3 doses (Loading), followed by 10 mg/kg of the study drug every other week×5 doses (Maintenance) (Cohort 2).

Subjects in both cohorts completed the following periods of assessment: Screening, Treatment, and Follow-Up. An overview of the cohorts is provided in Table 4.

Route of administration: IV.

TABLE 4
Cohort Overview.

| Cohort | No. of | Study drug | No. of | Frequency |
|---|---|---|---|---|
| No. | subjects | dose | doses | of doses |
| 1^{a} | Up to 8 | 10 mg/kg | 5 | Weekly |
| 2^{b} | Up to 12 | Loading: | 3 | Weekly |
| | | 30 mg/kg | | |
| | | Maintenance: | 5 | Every |
| | | 10 mg/kg | | other week |

^{a}Up to 3 subjects with pemphigus foliaceus are enrolled.
^{b}Two or fewer subjects with pemphigus foliaceus are enrolled.

Concomitant Medications and Procedures

All pemphigus treatments a subject receives within at least 3 months prior to enrollment and all other treatments a subject receives within 14 days prior to enrollment through the end of the study were documented.

Permitted Medications: (1) topical antibiotics to treat active infections that occurred during the study; (2) topical or systemic treatments for oral candidiasis; (3) topical lidocaine for transient pain relief as needed; (4) concomitant treatment that was medically indicated for any AEs the subject experienced during the study; (5) medication for potential infusion-related reactions (IRRs), including post-infusion headache: prophylactic use of acetaminophen, IV hydration, diphenhydramine, histamine2 (H2) blockers (e.g., ranitidine, famotidine); (6) low-strength topical corticosteroids (e.g., hydrocortisone <1%) applied to a single lesion contributing <10% of the PDAI total activity score; (7) topical tacrolimus, sirolimus or pimecrolimus applied to a single lesion contributing <10% of the PDAI total activity score; (8) dexamethasone elixir solution for oral lesions if dose remained stable throughout trial participation (swish and spit only); and (9) stable regimen of the following systemic immunosuppressants: azathioprine, mycophenolate mofetil, low-dose methotrexate, dapsone, cyclosporine, tacrolimus, sirolimus, corticosteroids, or low dose oral cyclophosphamide (<100 mg/day). Concomitant medications and treatments for co-existing conditions, including those for pemphigus, were permitted if not listed as prohibited.

Use of the following medications were not permitted during the study unless specified above as permitted: (1) rituximab or other anti-CD20 antibody; (2) monoclonal antibodies other than study drug; (3) any topical or systemic immunosuppressive drugs apart from those that are listed as permitted; (4) IV corticosteroids prior to infusion (except in subjects who received corticosteroids for treatment of a prior infusion reaction to the study drug); (5) any investigational drug or device; and (6) vaccinations within 2 weeks of screening through 28 days following final dose of study drug.

Corticosteroids

Before enrollment: Corticosteroids taken for pemphigus or any other condition prior to screening must be at a dose <1 mg/kg and the dose level must have not increased by more than 50% in the 2 weeks prior to screening. No pulse dosing of steroids was permitted in the 2 weeks prior to screening.

From screening until 2 weeks after the last dose of the study drug: The dose of corticosteroids taken for pemphigus or any other condition should remain stable (<10% change in dose level) from screening until 2 weeks after the last dose of the study drug. Corticosteroids should neither be started nor discontinued during this period with the exception of subjects who experienced an IRR that required corticosteroids as part of the management of the IRR. Such subjects may have received corticosteroids prophylactically prior to subsequent study drug infusions.

From 2 weeks after the last dose of the study drug until end of study participation: Only after at least 2 weeks beyond the last dose of the study drug, a slow corticosteroid taper may be started as per the following suggested schedule: if on >30 mg of prednisone per day, decrease by no more than 10 mg every two weeks until a final dose. If the subject would benefit from a change to the pemphigus treatment beyond the allowed steroid taper, this was considered on a case-by-case basis.

Laboratory Testing

Laboratory testing (hematology, urinalysis, serum chemistry, virology, serology, pregnancy tests, PD, PK, and ADAs) was performed using established methods by a central laboratory. Clinical safety laboratory panels tested in the study are listed in Table 5.

TABLE 5
Clinical Safety Laboratory Panels.

| Hematology | Serum Chemistry | Urinalysis |
|---|---|---|
| CBC with | Albumin | Appearance |
| differential and | Alkaline phosphatase | Color |
| blood smear | ALT | pH |
| Erythrocyte | AST | Specific gravity |
| sedimentation rat | BUN | Ketones |
| | C-Reactive Protein | Protein |
| | Calcium | Glucose |
| | Carbon dioxide | Nitrite |
| | Chloride | Urobilinogen |
| | Creatinine | Blood/hemoglobin |
| | Glucose | Leukocyte esterase |
| | LDH | Bilirubin |
| | Phosphorus | Microscopic examination |
| | Potassium | of sediment: only if the |
| | Sodium | results of the urinalysis |
| | Total and direct bilirubin | dipstick evaluation are |
| | Total protein | positive for |
| | Uric acid | blood/hemoglobin |

ALT = alanine aminotransferase; AST = aspartate aminotransferase; BUN = blood urea nitrogen; CBC = complete blood count; HIV = human immunodeficiency virus; LDH = lactate dehydrogenase; VZV = Varicella Zoster virus.

Pharmacokinetics (PK) Sampling

The following PK parameters were determined in Cohort 1: t_{1/2}, Cₘₐₓ, Tₘₐₓ, AUC₀₋₂₄, and AUC_{0-∞}. For Cohort 2, the PK parameters studied were Cₘₐₓ and Tₘₐₓ. For Cohort 2, the PK parameters determined were maximum serum concentration of the study drug and the associated Tₘₐₓ.

Pharmacodynamic Sampling

PD samples were collected for analyses throughout the study. Measurements for albumin were derived from the clinical safety laboratory results. Samples for each type of PD marker were collected according to the schedule shown in Table 6.

TABLE 6
Pharmacodynamic Assessments. Urine
IgG was collected in Cohort 1 only.

| | Collection Time Points | |
|---|---|---|
| Parameter | Cohort 1 | Cohort 2a |
| Immunoglobulins: IgG, | Screening and Days | Screening and Days |
| IgG subtypes (IgG1-4), | 0, 1, 2, 5, 7, 12, | 0, 7, 14, 28, 42, |
| IgA, IgM | 14, 19, 21, 28, 29, | 56, 70, 84, 91, |
| | 30, 33, 42, 56, | 112, and 140 |
| | 84, and 112 | |
| Circulating immune | Days 0, 5, 7, 12, | Days 0, 7, 14, 28, |
| complexes | 14, 19, 21, 28, 33, | 42, 56, 70, 84, |
| (CIC) | 42, 56, 84, and 112 | 91, 112, and 140 |
| Albumin | Screening and Days | Screening and Days |
| | 0, 7, 14, 21, 28, | 0, 7, 14, 28, 42, |
| | 33, 42, 56, 84, | 56, 70, 84, 91, |
| | and 112 | 112 and 140 |
| Anti-Dsg (1 and 3) | Screening and Days | Screening and Days |
| antibody titers | 0, 7, 14, 33, 56, | 0, 7, 14, 28, 42, |
| | 84, and 112 | 56, 70, 84, 91, |
| | | 112 and 140 |
| C3 and AECA | Days 0, 14, 33, 56, | Screening and Days |
| levels by indirect | 84, and 112 | 0, 7, 14, 28, 42, |
| immunofluorescence | | 56, 70, 84, 91, |
| | | 112 and 140 |
| Exploratory biomarkers | Days 0, 14, 33, 56, | Days 0, 28, and 91 |
| (RNAseq, urine IgG) | 84, and 112 | |
| Immunophenotyping | Days 0, 28, and 56 | Days 0, 28, and 91 |
| by flow cytometry for | | |
| measurement of T cells, | | |
| monocytes, NK cells, | | |
| and B cells | | |
| Exploratory biomarker | Day 0 | Day 0 |
| (FCGR2A SNP, via | | |
| buccal swab) | | |
| Exploratory pemphigus | Days 0, 5, 7, 12, 14, | Days 0, 7, 14, 28, |
| immune response | 19, 21, 28, 33, 42, | 42, 56, 70, 84, 91, |
| biomarkers | 56, 84, and 112 | 112, and 140 |

Pemphigus Disease Area Index (PDAI)

Pemphigus severity and disease activity were measured using the PDAI in regions where a validated questionnaire was available. A PDAI total activity score was determined at screening. To be eligible for study participation, the patient's grade by disease severity must have been >4. Assuming subject eligibility, the PDAI was administered during Treatment Period and Follow-up Period. Disease severity categories by PDAI are mild (0 to 8), moderate (9 to 24), and severe (≥25) (Shimizu et al., J Dermatol. 2014; 41(11):969-73). PDAI scores are determined as follows: 0 to 250 points for disease activity (≤120 for skin, ≤10 for scalp, and ≤120 for mucosa), and 0 to 13 points for damage (≤12 for skin and ≤1 for scalp) (Rosenbach et al., J Invest Dermatol. 2009; 129(10):2404-10).

Health-Related Quality of Life Assessments

For Cohort 2, health-related quality of life was assessed using ABQoL and Skindex-29 in regions where a validated questionnaire was available. The ABQoL questionnaire was developed in Australia as a patient-based measure to quantify disease burden, monitor disease activity and evaluate response to therapeutic intervention in patients with autoimmune bullous disease (Sebaratnam et al., JAMA Dermatol. 2013; 149(10):1186-91; Sebaratnam et al., Qual Life Res. 2015; 24(9):2257-60). Skindex-29 was developed to measure the effects of skin disease on patients' quality of life using a self-administered 30-question dermatology survey (Chren et al., J Invest Dermatol. 1996; 107(5):707-13.).

Statistical Considerations

Three populations were employed in the analysis of study data: (1) the Safety population consisted of all subjects who received at least one dose of study drug; (2) the PD population consisted of all subjects who received at least one dose of study drug and have post-dose PD data available; and (3) the PK population consisted of all subjects who received at least one dose of study drug and have post-dose PK data available. Primary safety analyses were performed on the Safety population. Demographics, subject disposition, screening, and baseline characteristics were summarized for the Safety, PD and PK populations, where appropriate.

Sample size. Formal sample size calculations were not performed. The number of subjects was chosen based on feasibility and was considered sufficient to meet the study objectives.

Criteria for Evaluation

Baseline analysis. Baseline characteristics included medical history, physical examination, vital signs, and ECG and were summarized using descriptive statistics by dose, dose regimen, and visit.

Safety analysis. The evaluation of the study drug based on vital signs, physical examination, ECGs, clinical safety laboratory tests, the incidence of AEs, TEAEs, and SAEs was summarized by dose and dose regimen, severity, and relationship to study drug.

Dose-finding analysis. The evaluation of the study drug based on total IgG levels and responses on the PDAI total activity score from baseline was summarized using descriptive statistics by dose and dose regimen, visit and time point, as applicable.

Statistical Methodology

Treatment-emergent AEs (TEAEs) were summarized using the Medical Dictionary for Regulatory Activities (MedDRA®; Version 19 or higher) System Organ Class (SOC) and preferred term, classified from verbatim terms. The incidence and percentage of subjects with at least one occurrence of a preferred term were included, using the most severe grade. The number of events per preferred term was also summarized. Causality (relationship to study drug [related/not related]) was summarized separately. TEAEs, SAEs, and AEs leading to withdrawal, dose modification, or treatment discontinuation were listed by subject and dose using SOC and preferred terms. Duration of AEs was determined and included in listings, along with action taken and outcome.

Laboratory results were summarized by time point, dose, and dose regimen. The incidence of laboratory abnormalities was summarized. The worst study grade after the first dose of study drug was summarized. Results for variables that are not coded were presented in the listings as below, within, or above the normal limits of the central laboratory. Vital sign measurements and change from baseline were summarized at each scheduled time point using descriptive statistics. PD/PK results were summarized by dose and dosing regimen. Descriptive statistics of PD/PK parameters for the study drug included mean, standard deviation (SD), coefficient of variation (CV), median, minimum, and maximum.

Immunogenicity results were summarized by cohort, visit and time point. Descriptive statistics included mean, SD, CV, median, minimum, and maximum.

Disease activity marker results were summarized by dose, dose regimen, and visit. Descriptive statistics included mean, SD, median, minimum, and maximum.

PDAI results were summarized by score (total activity score, total damage score), cohort, and visit. Descriptive statistics included absolute change from baseline and percent change from baseline.

Results for Cohort 1 Participants

Cohort and Treatments Administered

All eight subjects enrolled in Cohort 1 were administered all five weekly infusions during the study as planned in the protocol. Out of the eight patients, four subjects completed the study. The remaining four were withdrawn from the study due to physician decision (three patients) or due to receiving prohibited concomitant medication (one patient). The four subjects who withdrew early from the study were considered 100% compliant as they had not missed any doses and withdrew from the study during the Follow-up Period. No infusion interruption, study drug discontinuation, dose reduction or study discontinuation due to AEs was reported for any subject. Out of the four patients that withdrew, two patients withdrew on Day 34 (5 days after receiving the last dose of the study drug on Day 29), one patient withdrew on Day 78 (49 days after receiving the last dose of the study drug on Day 29), and one patient withdrew on Day 85 (56 days after receiving the last dose of the study drug on Day 29).A summary of the cohort's demographics can be found in Table 7.

TABLE 7
Summary of demographics.

| | | Count of | Percentage of |
|---|---|---|---|
| | | participants | participants |
| Age | Mean: 51.4 years | 8 | |
| | Standard deviation: | | |
| | 16.4 years | | |
| Sex | Female | 5 | 62.5% |
| | Male | 3 | 37.5% |
| Ethnicity | Hispanic or Latino | 0 | 0% |
| | Not Hispanic or | 8 | 100% |
| | Latino | | |
| | Unknown or not | 0 | 0% |
| | reported | | |
| Race | American Indian | 0 | 0% |
| | or Alaska Native | | |
| | Asian | 0 | 0% |
| | Native Hawaiian | 0 | 0% |
| | or other Pacific | | |
| | Islander | | |
| | Black or African | 3 | 37.5% |
| | American | | |
| | White | 5 | 62.5% |
| | More than one race | 0 | 0% |
| | Unknown or not | 0 | 0% |
| | reported | | |
| Type of pemphigus | Vulgaris | 7 | 87.5% |
| | Foliaceus | 1 | 12.5% |
| Age at diagnosis of | Mean: 41.3 years | | |
| pemphigus | Standard deviation: | | |
| | 18.9 years | | |
| Pemphigus disease | Mean: 9.98 | | |
| duration since | Standard deviation: | | |
| Day 0 visit (years) | 0.699 | | |
| Type of tissue-based | Biopsy | 7 | 87.5% |
| test positive for | Direct | 1 | 12.5% |
| pemphigus | immunofluorescence | | |
| | (DIF) | | |
| Tissue-based test | Mean: 9.65 | | |
| positive for | Standard deviation: | | |
| pemphigus duration | 10.553 | | |
| since Day 0 | | | |
| visit (years) | | | |
| Current | Mean: 5.1 | | |
| exacerbation since | Standard deviation: | | |
| Day 0 visit | 3.87 | | |
| (months) | | | |
| Baseline BMI | Mean: 31.48 | | |
| (kg/m2) | Standard deviation: | | |
| | 4.917 | | |

Serum Concentration of the Study Drug

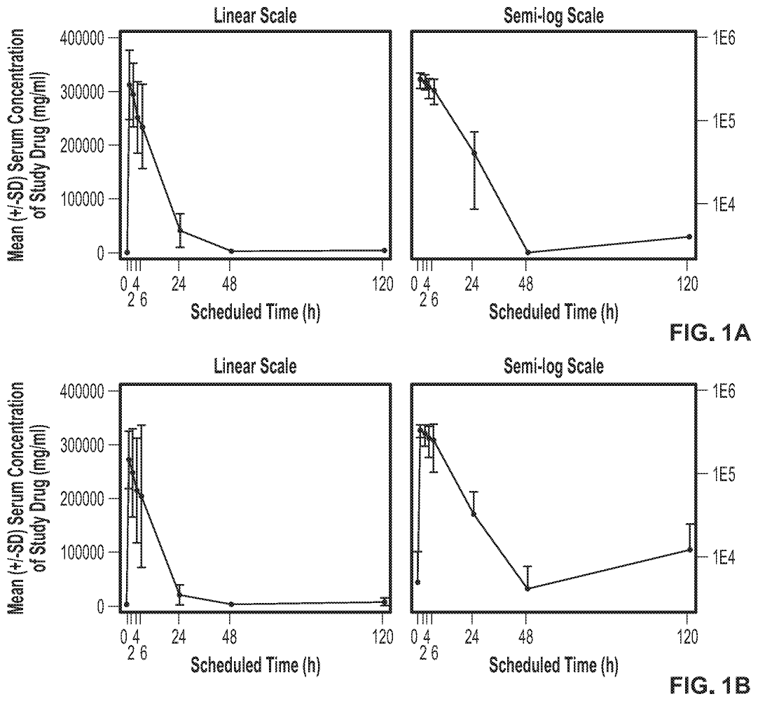

The mean serum concentration-time profiles for the study drug (linear scale and semi-logarithmic scale) following 1-hour infusion of 10 mg/kg of the study drug on Days 0 and 28 are shown in FIG. 1. A summary of PK parameters (untransformed) of the study drug following 1-hour infusion of 10 mg/kg on Day 0 and Day 28 is provided in Table 8. The mean Cₘₐₓ decreased from 313.1 μg/mL on Day 0 to 292.1 μg/mL on Day 28. AUC₀₋ₗₐₛₜ was 3727 h*μg/mL on Day 0 and 3220 h*μg/mL on Day 28 (Table 8). There was no apparent accumulation of the study drug after 5 weekly doses at 10 mg/kg IV as indicated by Cₘₐₓ (292.1 μg/mL) and AUC₀₋ₗₐₛₜ (3220 h*μg/mL).

TABLE 8
Pharmacokinetic Parameter Estimates in Subjects
With Pemphigus (Cohort 1, 10 mg/kg)

| | | | | |
|---|---|---|---|---|
| | Cₘₐₓ | tₘₐₓ | t_{1/2} | AUC₀₋ₗₐₛₜ |
| Parameter | (μg/mL) | (h) | (h) | (h*μg/mL) |

Day 0

| | | | | |
|---|---|---|---|---|
| N | 8 | 8 | 6 | 8 |
| Mean | 313.1 | 2.326 | 6.035 | 3726.8 |
| SD | 63.72 | 2.0652 | 2.0751 | 1279.85 |
| CV % | 20.4 | 88.8 | 34.4 | 34.3 |
| Min | 230 | 1.05 | 4.16 | 2093 |
| Median | 315.5 | 1.170 | 5.186 | 3514.6 |
| Max | 408 | 6.97 | 9.43 | 6203 |
| Geometric mean | 307.5 | NA | NA | 3545.9 |
| Geometric CV % | 20.6 | NA | NA | 34.6 |

Day 28

| | | | | |
|---|---|---|---|---|
| N | 8 | 8 | 2 | 8 |
| Mean | 292.1 | 1.859 | 7.851 | 3220.4 |
| SD | 93.03 | 2.0804 | 4.2225 | 1501.51 |
| CV % | 31.8 | 111.9 | 53.8 | 46.6 |
| Min | 204 | 1.03 | 4.87 | 1884.0 |
| Median | 260.0 | 1.080 | 7.851 | 2537.2 |
| Max | 488 | 7.00 | 10.84 | 6190 |
| Geometric mean | 281.3 | NA | NA | 2962.6 |
| Geometric CV % | 28.7 | NA | NA | 44.3 |

IgG, IgG Subtypes, IgA, IgM

Mean total IgG levels were reduced a maximum of 57.3% from baseline by Day 30 (after the fifth weekly dose), with recovery throughout the remainder of the study.

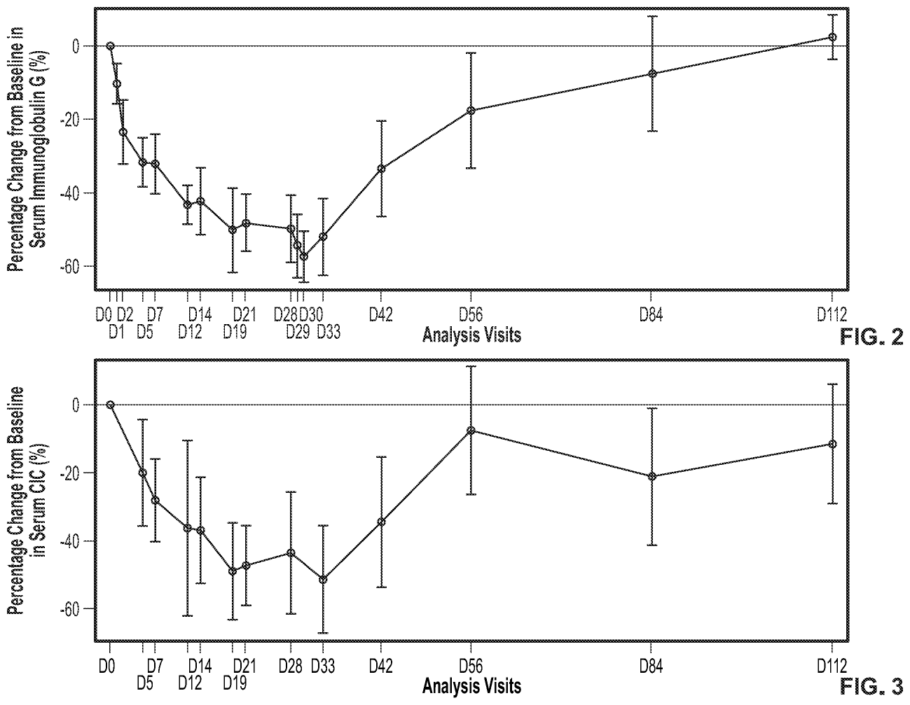

A decrease in serum total IgG concentration was observed through Day 30 after administration (n=8). Mean serum total IgG levels reached a maximum reduction of 57.3% from baseline by Day 30, following 5 weekly doses of the study drug at 10 mg/kg. By Day 56, mean total IgG levels had recovered to 17.5% below baseline (6/8 subjects), and continued to increase for those subjects (4/8 subjects) who reached the Day 112 final follow-up visit (FIG. 2). A nadir of mean percent change from baseline for IgG subclasses was also consistently achieved at Day 30. Approximately 14% decrease in mean urine IgG concentration was observed through Day 112 (n=4).

There were no meaningful changes in levels of IgA or IgM. The mean percent change from baseline for serum IgA ranged between 0.1% (Day 7) and 15.4% (Day 84). The mean percent change from baseline for serum IgM ranged between 7.4% (Day 42) and 10.5% (Day 112).

Circulating Immune Complexes (CIC)

Circulating immune complexes were assessed using the C1Q binding assay and the Raji cell immune complex assay. The Raji assay assesses the binding of ICs to the complement receptors on the cell surface of the Raji cells. By contrast the C1Q assay is immobilized in a solid phase. As evidenced by the serum C1Q binding assay, at Day 33 (n=8), mean circulating IgG IC levels reached a nadir of 51.4% below baseline, and returned to baseline by Day 56. The mean percentage decrease in CIC was 34% on Day 42 (n=6), 21% on Day 84 (n=5), and 11% on Day 112 (n=4). As evidenced by the Raji cell immune complex assay, a nadir of mean percent change from baseline for CIC was 21% on Day 42 (n=5). The mean percentage of CIC started increasing on Day 56. Plots of percent change from baseline in CIC by the serum C1Q binding assay and by the Raji cell immune complex assay are presented in FIG. 3.

Albumin

No clinically meaningful alterations in albumin levels were observed after administration of the study drug. The albumin levels in all subjects in Cohort 1 were within the reference range, except for 1 subject; one subject had borderline low levels of albumin at the Day 7 and 14 visits.

Anti-Dsg1 and Anti-Dsg3 Antibodies

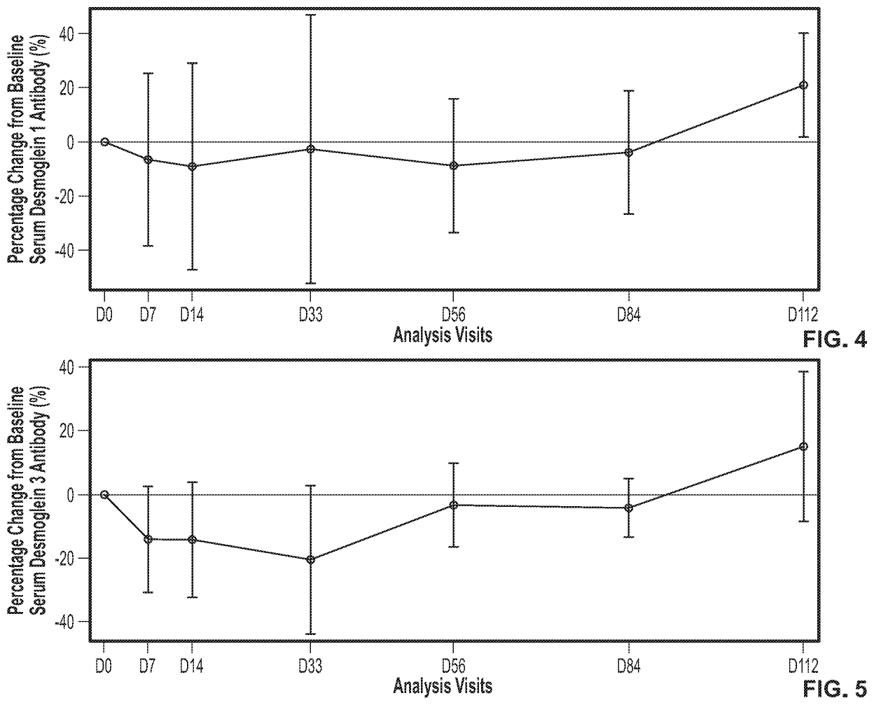

Mean anti-Dsg1 and anti-Dsg3 antibody levels decreased following study drug administration as shown in FIGS. 4 and 5. At baseline, the mean (SD) anti-Dsg1 antibody level was 53.9 (42.61) U/mL. At Day 33 (n=7), the mean (SD) anti-Dsg1 antibody level was 63.1 (62.77) U/mL (2.5% reduction from baseline). At Day 56 (n=5), the mean (SD) anti-Dsg1 antibody level was 51.2 (62.66) U/mL (8.7% reduction from baseline). At baseline, the mean (SD) anti-Dsg3 antibody level was 120.9 (67.71) U/mL (n=8). At Day 33 (n=7), the mean (SD) anti-Dsg3 antibody level was 95.6 (66.39) U/mL (20.4% reduction from baseline). At Day 56 (n=5), the mean (SD) anti-Dsg3 antibody level increased to 114.0 (81.22) U/mL (3.3% reduction from baseline). These results indicate that the study drug effectively decreases levels of IgG and CICs and lowers the levels of autoantibodies against Dsg 1 and 3, primary antigenic targets of pathogenic autoantibodies in pemphigus disease.

C3 and Anti-Epithelial Cell Antibodies (AECAs)

Complement component (C3) levels for all subjects in Cohort 1 were within the normal reference range (90-180 mg/dL) throughout the duration of the study. All subjects in Cohort 1 had negative screening tests for anti-basement membrane zone (BMZ) antibodies throughout the duration of the study. All 8 subjects in Cohort 1 had positive anti-intercellular substance (ICS) screening tests at some point during the study. Seven of these 8 subjects also had positive ICS titers at some point during the study.

Analysis of Immunogenicity

A total of 7/8 [87.5%] of subjects developed ADAs after treatment with study drug IV at 10 mg/kg (Cohort 1): 4 subjects at Day 14 (n=8) with titer range of 2 to 18, 7 subjects at Day 28 (n=8) with titer range of 6.6 to 1560, and 5 subjects at Day 56 (n=6) with titer range of 45.5 to 2140. Of the 4 subjects who completed the study and had ADAs assessed at Day 112, 3 were ADA-positive. The presence of ADAs does not appear to have a significant impact on the PK or PD of the study drug. No apparent impact on the IgG lowering effect of the study drug was observed in association with the appearance of ADAs. For the 4 subjects who developed ADAs at Day 14, IgG levels continued to decrease with dosing of the study drug. Individual and mean drug exposure as measured by AUC did not decrease significantly at Day 28. For subjects who developed ADAs at Day 14, IgG levels continued to decrease with dosing of the study drug. One subject with a high ADA titer of 1560 on Day 28 experienced two Grade 2 infusion-related reactions after the fourth and fifth weekly dose, respectively. However, high ADA titer was not necessarily associated with infusion-related reactions.

Pemphigus Disease Area Index (PDAI) Score

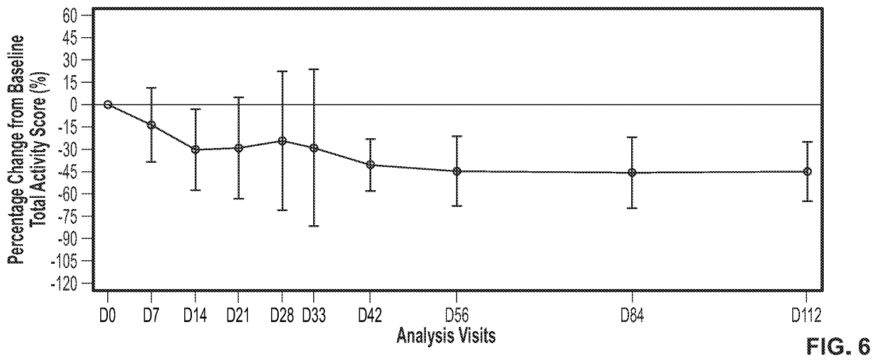

A consistent reduction in the PDAI total activity score after administration of the study drug was observed across all visits. A nadir of mean percent change from baseline for PDAI total activity score was approximately 45.7% and was achieved at the Day 84 visit (FIG. 6).

Safety Analysis

In general, the study was well tolerated in subjects with pemphigus at 10 mg/kg administered IV. There were no deaths, or TEAEs leading to discontinuation, interruption, or reduction of study drug. A total of 20 related TEAEs (all Grade 1 and 2) were reported among 7 subjects. The most common related TEAE was Grade 1 headache at 75%, which resolved with or without treatment. There were 2 reported SAEs (disease progression and acute kidney injury) in 1 subject that were assessed as not related to the study drug. One subject developed infusion-related reactions at Doses 4 and 5 which presented as wheals and itching and resolved with treatment with oral diphenhydramine 25 mg. This subject had a high ADA titer at the time of the infusion-related reactions.

1. A method of treating pemphigus and/or a pemphigoid disease in a subject in need thereof, the method comprising administering to the subject an FcRn inhibitor, wherein the FcRn inhibitor is administered at a dose of at least 10 mg/kg of the subject's body weight.

2. The method according to claim 1, wherein the FcRn inhibitor is administered at a dose of at least 10 mg/kg of the subject's body weight once a week for at least five weeks.

3. The method according to claim 1, wherein the FcRn inhibitor is administered at a dose of 10 mg/kg of the subject's body weight.

4. The method according to claim 1, wherein the FcRn inhibitor is administered once a week for five weeks.

5. The method according to claim 1, wherein the pemphigus is pemphigus vulgaris, pemphigus foliaceus, paraneoplastic pemphigus, drug-induced pemphigus, endemic pemphigus (fogo selvagem), pemphigus erythematosus (Senear-Usher syndrome), or pemphigus vegetans.

6. (canceled)

7. (canceled)

8. The method according to claim 1, wherein the pemphigoid disease is bullous pemphigoid, mucous membrane pemphigoid, pemphigoid gestationis, epidermolysis bullosa acquisita, anti-laminin g1/anti-p200 pemphigoid, or lichen planus pemphigoid.

9. The method according to claim 1, wherein the subject:
a. has been diagnosed with pemphigus vulgaris or foliaceus based on (i) a clinical history consistent with pemphigus vulgaris or foliaceus, (b) presence of anti-Dsg 1 or anti-Dsg3 antibodies above the upper limit of normal, and/or (c) a history of at least one positive tissue-based test for pemphigus vulgaris or foliaceus;
b. experiences active pemphigus vulgaris or foliaceus and has (i) lesions lasting longer than two weeks, and/or (ii) at least three active lesions in skin or mucosa or at least two active lesions, wherein at least one of the at least two active lesions is a skin lesion with a diameter of at least 1 cm; and/or
c. exhibits a Pemphigus Disease Area Index (PDAI) total activity score of at least four.

10. The method according to claim 1, the method further comprising:
a. measuring a level of IgG for the subject, wherein administering the FcRn inhibitor leads to a decrease in IgG level;
b. measuring a level of circulating immune complexes (CIC) for the subject, wherein administering the FcRn inhibitor leads to a decrease in CIC level;
c. measuring the PDAI total activity score for the subject, wherein administering the FcRn inhibitor leads to a decrease in PDAI total activity score;
d. measuring an anti-Dsg1 antibody titer for the subject, wherein administering the FcRn inhibitor leads to a decrease in anti-Dsg1 antibody titer;
e. measuring an anti-Dsg3 antibody titer for the subject, wherein administering the FcRn inhibitor leads to a decrease in anti-Dsg3 antibody titer;
f. measuring an anti-epithelial cell antibody (AECA) titer for the subject, wherein administering the FcRn inhibitor leads to a decrease in AECA titer; or
g. measuring a complement component 3 (C3) level for the subject, wherein administering the FcRn inhibitor leads to a decrease in the C3 level.

11. The method according to claim 1, wherein the subject exhibits one or more of the following conditions and wherein the administration of the FcRn inhibitor reduces the occurrence of one or more of the following conditions:
a. fluid-filled skin blisters;
b. ruptured blisters;
c. scaly, inflamed, painful patches on the skin;
d. burning, pain, and itching at the site of the blisters; and/or
e. chronic skin infections due to ruptured and irritated blisters.

12. The method according to claim 1, wherein the FcRn inhibitor is an anti-FcRn antibody or antigen-binding fragment thereof, wherein the anti-FcRn antibody or antigen-binding fragment thereof comprises a heavy chain variable region comprising a CDR1, CDR2, and CDR3 (HCDR1, HCDR2 and HCDR3) and a light chain variable region comprising a CDR1, CDR2, and CDR3 (LCDR1, LCDR2 and LCDR3); and wherein:
a. HCDR1 comprises the amino acid sequence of SEQ ID NO: 3; HCDR2 comprises the amino acid sequence of SEQ ID NO: 4; HCDR3 comprises the amino acid sequence of SEQ ID NO: 5; LCDR1 comprises the amino acid sequence of SEQ ID NO: 6; LCDR2 comprises the amino acid sequence of SEQ ID NO: 7; and LCDR3 comprises the amino acid sequence of SEQ ID NO: 8;
b. HCDR1 comprises the amino acid sequence of SEQ ID NO: 11 or SEQ ID NO: 12; HCDR2 comprises the amino acid sequence of SEQ ID NO: 13 or SEQ ID NO: 14; HCDR3 comprises the amino acid sequence of SEQ ID NO: 15, SEQ ID NO: 16, SEQ ID NO: 17, SEQ ID NO: 18, or SEQ ID NO: 19; LCDR1 comprises the amino acid sequence of SEQ ID NO: 20; LCDR2 comprises the amino acid sequence of SEQ ID NO: 21; and LCDR3 comprises the amino acid sequence of SEQ ID NO: 22;
c. HCDR1 comprises the amino acid sequence of SEQ ID NO: 11; HCDR2 comprises the amino acid sequence of SEQ ID NO: 13; HCDR3 comprises the amino acid sequence of SEQ ID NO: 19; LCDR1 comprises the amino acid sequence of SEQ ID NO: 20; LCDR2 comprises the amino acid sequence of SEQ ID NO: 21; and LCDR3 comprises the amino acid sequence of SEQ ID NO: 23 or SEQ ID NO: 24;
d. HCDR1 comprises the amino acid sequence of SEQ ID NO: 25; HCDR2 comprises the amino acid sequence of SEQ ID NO: 26; HCDR3 comprises the amino acid sequence of SEQ ID NO: 27; LCDR1 comprises the amino acid sequence of SEQ ID NO: 28; LCDR2 comprises the amino acid sequence of SEQ ID NO: 29; and LCDR3 comprises the amino acid sequence of SEQ ID NO: 30;
e. HCDR1 comprises the amino acid sequence of SEQ ID NO: 31; HCDR2 comprises the amino acid sequence of SEQ ID NO: 32; HCDR3 comprises the amino acid sequence of SEQ ID NO: 33; LCDR1 comprises the amino acid sequence of SEQ ID NO: 34; LCDR2 comprises the amino acid sequence of SEQ ID NO: 35; and LCDR3 comprises the amino acid sequence of SEQ ID NO: 36;
f. HCDR1 comprises the amino acid sequence of SEQ ID NO: 37; HCDR2 comprises the amino acid sequence of SEQ ID NO: 38; HCDR3 comprises the amino acid sequence of SEQ ID NO: 39; LCDR1 comprises the amino acid sequence of SEQ ID NO: 40; LCDR2 comprises the amino acid sequence of SEQ ID NO: 41; and LCDR3 comprises the amino acid sequence of SEQ ID NO: 42; or
g. HCDR1 comprises the amino acid sequence of SEQ ID NO: 43; HCDR2 comprises the amino acid sequence of SEQ ID NO: 44; HCDR3 comprises the amino acid sequence of SEQ ID NO: 19; LCDR1 comprises the amino acid sequence of SEQ ID NO: 20; LCDR2 comprises the amino acid sequence of SEQ ID NO: 45; and LCDR3 comprises the amino acid sequence of SEQ ID NO: 23.

13. The method according to claim 12, wherein HCDR1 comprises the amino acid sequence of SEQ ID NO: 3; HCDR2 comprises the amino acid sequence of SEQ ID NO: 4; HCDR3 comprises the amino acid sequence of SEQ ID NO: 5; LCDR1 comprises the amino acid sequence of SEQ ID NO: 6; LCDR2 comprises the amino acid sequence of SEQ ID NO: 7; and LCDR3 comprises the amino acid sequence of SEQ ID NO: 8.

14. The method according to claim 1, wherein the FcRn inhibitor is an anti-FcRn antibody or antigen-binding fragment thereof comprising a heavy chain variable region and a light chain variable region, wherein:
the heavy chain variable region comprises the sequence of SEQ ID NO: 1, or a sequence that is at least 80% identical to the sequence of SEQ ID NO: 1; and
the light chain variable region comprises the sequence of SEQ ID NO: 2, or a sequence that is at least 80% identical to the sequence of SEQ ID NO: 2.

15. The method according to claim 14, wherein the heavy chain variable region comprises the sequence of SEQ ID NO: 1 and the light chain variable region comprises the sequence of SEQ ID NO: 2.

16. The method according to claim 1, wherein the FcRn inhibitor is an anti-FcRn antibody or antigen-binding fragment thereof comprising a heavy and a light chain, wherein:
the heavy chain comprises the amino acid sequence of SEQ ID NO: 9, or a sequence that is at least 80% identical to a sequence of SEQ ID NO:9; and
the light chain comprises the amino acid sequence of SEQ ID NO: 10, or a sequence that is at least 80% identical to a sequence of SEQ ID NO: 10.

17. The method according to claim 16, wherein the heavy chain comprises the amino acid sequence of SEQ ID NO: 9 and the light chain comprises the amino acid sequence of SEQ ID NO: 10.

18. The method according to claim 1, wherein the FcRn inhibitor is an Fc region, or FcRn-binding fragment thereof, and wherein the Fc region comprises the amino acid sequence of SEQ ID NO:46, SEQ ID NO:47, or SEQ ID NO:48.
